(19) United States
(12) Patent Application Publication
(10) Pub. No.: US 2006/0264858 A1
(43) Pub. Date: Nov. 23, 2006

(54) Multi-functional training garment

(76) Inventors: Donald Carroll Roe, West Chester, OH (US); Cynthia Jean Panning, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/133,973

(22) Filed: May 20, 2005

(51) Int. Cl. A61F 13/15

(52) U.S. Cl. 604/361

(57) ABSTRACT

An absorbent article including features facilitating toilet training of a wearer is disclosed. In particular, the absorbent articles may have one or several of a variety of multi-functional training features. These features include features designed to facility ease of application and removal of the garment such as informational graphics or slow recovery elasticized features. Other features may be designed to provide training feedback to a wearer upon a urination incident or upon remaining dry for a period of time. These features may be employed in combination in a multi-functional article to provide both multi-sensory feedback and ease of application and removal features.

### FIELD OF THE DISCLOSURE

The present disclosure is applicable to absorbent articles including diapers, training pants, pull-on diapers, incontinence briefs, incontinence undergarments, absorbent inserts, diaper holders and liners, and the like. This disclosure is particularly related to absorbent articles having combinations of features suited for training purposes, in particular for urinary toilet training and/or teaching of dressing.

### BACKGROUND OF THE DISCLOSURE

Absorbent articles are well known in the art. These articles typically have an absorbent assembly held or positioned in proximity to the body of a wearer during use in order to capture and absorb bodily exudates discharged from the wearer. Typical absorbent articles include a topsheet facing the wearer, which permits fluid exudates to pass through, and a backsheet, which prevents the exudates from escaping from the absorbent article.

Disposable absorbent articles such as diapers are designed to absorb and contain bodily waste in order to prevent soiling of the body and clothing of the wearer. Disposable diapers typically comprise a single design available in different sizes to fit a variety of wearers ranging from newborns to toddlers undergoing toilet training. The design of the diaper typically affects performance, such as the ability to absorb and contain bodily waste. The fit of the diaper on the wearer's body is typically affected by, for example, the size of the diaper waist opening, the size of the openings around the thighs, and the length or “pitch” of the diaper.

Typically, conventional diaper products for infants and small children have included a front waist portion, and a rear waist portion which are releasably connected about the hips of the user during use by fasteners such as adhesive tape fasteners or hook and loop type fasteners. Commonly such diapers are applied by laying the baby on its back, positioning the diaper between the baby's legs and fastening the fasteners about the waist.

More recently, there have been several prior art disposable absorbent articles of the “pull-on” or “pants” type. These articles are typically placed on a user in a closed waist configuration, and therefore are designed to be pulled up over the hips and buttocks of the user into position around the waist and between the legs. Ideally, application would be substantially accomplished by the child, however these articles are typically applied at least partially by the caregiver due to the difficulty involved. A caregiver will often perform or assist in one or more of the following actions: (1) threading the user's legs through leg holes in the article; (2) pulling the article over the user's hips and buttocks; and (3) correcting or adjusting the fit of the article once it is in position. Typically, such pull-on articles have a stretchable portion, such as a stretchable side panel which expands to allow the article to be pulled over the hips and then elastically retracts to provide a conforming fit of the article. A variation of the pull-on articles includes refastenable seamed areas, such as refastenable side seams. While these alternative articles may be opened by unfastening the seams, they are typically intended to be applied to a user while in the closed configuration, and therefore include a stretchable portion as noted above.

The art has made some attempts to improve both the fit of disposable absorbent garments for infants and small children and to make such articles easier for such children to don and remove with little to no assistance. U.S. Patent Application Ser. No. 60/647,246 describes a disposable pull-on type diaper having a low force, slow recovery elastic waist. Such a feature allows the garment to be stretched into an expanded configuration (such as an expanded waist opening) for ease of application by a child, while still allowing the waist to recover to provide a snug fit and anchoring of the product. The slower than conventional speed of the recovery of the elastics gives the child sufficient time to pull the garment over the hips and buttocks while the garment is still in at least a partially expanded configuration.

U.S. patent application Ser. No. 11/083,607 describes a pull on type wearable article with an informational image. Due to physiological, psychological, or other factors, most children, particularly in the 12-30 month age range, are naturally inclined to grab the most easily visible and accessible portion of the pull-on product, which is the front waist region. Because the pull-on article must be pulled over the buttocks and hips, the tendency to pull at the front of the product often leads to failure and frustration because this action increases the circumferential tension in the back of the diaper, causing it to lodge tightly at the bottom of the buttocks. Further, no vertical tension is applied to the area that could dislodge the article, which is the back waist region of the article. Accordingly, it is more advantageous for the child to grasp and pull the product from the sides, thereby distributing vertical pulling force to both the front and back regions. Articles described in the above-cited application may have printed “hand prints” or other indicia indicating to a child the optimal location to grab in order to most easily pull the product on. Such a printed “hand” or other feature may be placed on a location of increased stiffness or other feature of the garment which acts as a “handle” to allow a child to more easily don and remove the article. Both of the patent applications cited above are commonly owned by the assignee of the instant invention.

In addition to being concerned about ease of application and removal of disposable absorbent articles (especially of the pants type) by toddlers and young children, the art has also been concerned with assisting children and caregivers with the toilet training process. The toilet training stage may be referred to as the “point of exit” from the diaper product category because toddlers who have successfully completed toilet training typically no longer wear diapers. The age at which children are toilet trained in “developed” countries has increased steadily over the past several decades and is now in the range of about 24-48 months.

One reason for which toilet training has become delayed is that significant technical improvements have been made in diaper dryness and comfort. For example, when wearing a typical modern diaper, the child may have dry skin even after one or more occurrences of urination. As a result, the child may feel little or no discomfort and often may not even be aware that he or she has urinated. However, having the child feel discomfort following urination in his or her “pants” may assist with learning and/or provide motivation to learn to voluntarily retain urine. It is possible to use cloth training pants that leave the skin wet and, due to their high breathability, promote evaporative cooling of the skin, further enhancing discomfort. However, cloth training pants have poor urine containment, often leading to wet clothing and wet surroundings, e.g., carpeting, furniture, etc.

The art has described a number of approaches designed to facilitate the toilet training process. U.S. patent application Ser. No. 11/098,362 (also owned by the assignee of the present application) describes an absorbent article which incorporates a dryness indicating graphic to provide a positive feedback for children during toilet training. As described in this application, such dryness indicating graphics may be used in combination with features which provide tactile feedback in the event of urination such as a wetness sensation liner.

It has been found during development of the present invention that the skills of dressing and clothing removal and success at the toilet training process are related and are not necessarily independent of each other. For example, some children may development basic continence skills (recognizing the need to urinate, being able to postpone urination willingly, etc.) in advance of development of sufficient dressing and undressing skills to complete toilet training in an optimal manner. If a child is not able to remove clothing (such as a disposable absorbent garment) in a satisfactory manner, he or she may not be able to use the toilet prior to accidental soiling. Additionally, it has been found during development of the present invention that even frustration at putting on a new (or re-applying the previously worn) disposable absorbent garment can be an obstacle to toilet training. If a child cannot easily replace such a garment, he or she may hesitate to take it off in the first place. Such de-motivation to undress can lead to accidents even if the child may otherwise recognize the urge to urinate. Of course, children develop different skills at different rates and some children may develop increased dexterity in advance of basic urinary continence. Nevertheless, it has been found during development of the present invention that ease of application and removal of disposable garments and providing feedback to children are both related to successful toilet training. It would, therefore, be desirable to provide a multi-functional training garment which can assist children as their dexterity develops in applying and/or removing such garments, while also providing other toilet training feedback functionality.

### SUMMARY OF THE DISCLOSURE

The present invention is directed to a multi-functional training garment. Such a garment may include a front waist region having a pair of longitudinal sides, a rear waist region having a pair of longitudinal sides, and a crotch region connecting the front waist region and the rear waist region. The garment may also have a pair of front side panels extending outwardly from each of the longitudinal sides of the front waist region and a pair of rear side panels extending outwardly from each of the longitudinal sides of the rear waist region. The garment may include a pair of side seams joining each of the front side panels with a corresponding rear side panel so as to form a pant-like garment having a waist opening and a pair of leg openings. The garment may include at least one application or removal aid. Such an application or removal aid may be selected from the group consisting of an informational image feature, a slow recovery elasticized region feature, and refastenable side seams. The garment may further include at least one sensory feedback feature for training. The sensory feedback feature may be selected from the group consisting of appearing graphics, disappearing graphics, and a wetness sensation member.

The at least one application or removal aid may include an informational image. The informational image may communicate a pulling location in which to grip the article. The informational image may be located on one of the front side panels and the rear side panels. The informational image may comprise a hand graphic. Such a hand graphic may have size dimensions substantially corresponding to at least a portion of a child's hand, such as a lateral size dimension of about 1 to about 10 centimeters and a longitudinal size dimension of about 1 to about 5 centimeters.

The application or removal aid may comprise a slow recovery elasticized region. This region may comprise an elastic belt region disposed generally about the waist opening. The elastic belt region may exhibit less than about 50% of the maximum force after 15 seconds as measured by the Percent Release Test. The belt region may exhibit less than about 90% of the maximum force after 45 seconds as measured by the Percent Release Test. The elastic belt region may exhibit a 30% Recovery Time of at least 1 second. The elastic belt region may exhibit a recovery speed of less than about 508 mm/min.

The application or removal aid of the absorbent article may include a refastenable side seam. Such a refastenable side seam may comprise mechanical fasteners. The mechanical fasteners may comprise hook and loop type fasteners or hook to hook type fasteners The refastenable side seam may comprise cohesive fasteners or adhesive fasteners.

The sensory feedback feature of the absorbent article may comprise appearing graphics. Such appearing graphics may appear over time. The appearing graphics may be are less visible upon exposure to liquid. The sensory feedback feature may comprise disappearing graphics. The disappearing graphics may become less visible upon exposure to liquid.

The sensory feedback feature may comprise a wetness sensation member. The wetness sensation member may be optionally removable from said article by a user.

An absorbent article of the invention may include a front waist region having a pair of longitudinal sides, a rear waist region having a pair of longitudinal sides, and a crotch region connecting said front waist region and said rear waist region. The article may further include a pair of front side panels extending outwardly from each of the longitudinal sides of the front waist region and a pair of rear side panels extending outwardly from each of the longitudinal sides of the rear waist region. The article may also include a pair of side seams joining each of the front side panels with a corresponding rear side panel so as to form a pant-like garment having a waist opening and a pair of leg openings. Additionally, the absorbent article may include at least one application or removal aid and at least one sensory feedback feature for toilet training.

### BRIEF DESCRIPTION OF THE DRAWINGS

In the accompanying drawing figures, like reference numerals identify like elements, which may or may not be identical in the several exemplary embodiments that are depicted. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description.

FIG. 1a is a perspective view of a disposable pull-on diaper according to the present invention.

FIG. 1b is a perspective view of a disposable pull-on diaper according to the present invention showing a fastening system.

FIG. 2 is a perspective view of the disposable pull-on diaper of FIG. 1 laid flat in its unseamed, uncontracted state.

FIG. 3 is a perspective view of a disposable pull-on diaper according to the present invention.

FIG. 4 is a perspective view of the disposable pull-on diaper FIG. 3 laid flat in its unseamed, uncontracted state.

FIG. 5a is a plan view of a disposable absorbent article having a wetness sensation member disposed on a body-facing surface.

FIG. 5b is a cross sectional view of the disposable absorbent article shown in FIG. 5a illustrating the layers of the wetness sensation member.

FIG. 6a is a plan view of a disposable absorbent article having a wetness sensation member integrated with the topsheet.

FIG. 6b is a cross sectional view of the disposable absorbent article illustrated in FIG. 6a.

FIG. 7a is a front perspective view of an absorbent article having appearing graphics in an initial state.

FIG. 7b is a front perspective view of the absorbent article of FIG. 7a showing a first appearing graphic in a subsequent state.

FIG. 7c is a front perspective view of the absorbent article of FIG. 7a with both a first and a second appearing graphic in subsequent states.

FIG. 8a is a partial section view of an absorbent article with a single layer backsheet.

FIG. 8b is a partial section view of an absorbent article with a double layer backsheet.

FIGS. 9A and 9B are plan views of an informational image applied to a zero-strain laminate in the relaxed and extended states, respectively.

FIGS. 10A and 10B are plan views of an informational image applied to an elastomeric film in the relaxed and extended states, respectively.

FIGS. 11A and 11B are plan views of an informational image applied to a pre-stretch laminate in the relaxed and extended states, respectively.

FIG. 12 is an enlarged plan view of part of an article having a main portion with a projection on which the informational image is disposed.

FIG. 13 is an enlarged plan view of part of an article having a side panel with a projection on which the informational image is disposed.

FIG. 14 is an enlarged plan view of part of an article having an informational image spanning a seam of a side panel.

FIG. 15 is an enlarged plan view of an alternative informational image spanning a seam of a side panel.

FIG. 16 is a side elevational view, in cross-section, of an article having a texture feature on an exterior surface.

FIG. 17 is a side elevational view, in cross-section, of an article having an alterative embodiment of a texture feature on an exterior surface.

FIG. 18 is a side elevational view, in cross-section, of an article having an alternative embodiment of a texture feature on an exterior surface.

FIG. 19a shows the custom hooks used in Percent Release Test.

FIG. 19b shows the custom hooks used in the Percent Release Test with a sample engaged thereon.

FIG. 20 is a graph plotting the Percent Maximum Force versus time for the Examples.

FIG. 21 is a graph plotting the normalized force versus time for the Examples.

### DETAILED DESCRIPTION OF THE DISCLOSURE

Definitions

As used herein, the following terms have the following meanings:

The term “absorbent article” refers to a device that absorbs and contains liquid, and more specifically, refers to a device that is placed against or in proximity to the body of the wearer to absorb and contain the various exudates discharged from the body.

The term “disposable” refers to absorbent articles that generally are not intended to be laundered or otherwise restored or reused as absorbent articles, i.e., they are intended to be discarded after a single use and, preferably, to be recycled, composted or otherwise disposed of in an environmentally compatible manner.

The term “unitary” refers to an absorbent article that is formed of separate parts united together to form a coordinated entity so as to not require separate manipulative parts like a separate holder and liner.

The term “disposed” refers to an element being attached and positioned in a particular place or position in a unitary structure with other elements.

The term “diaper” refers to an absorbent article generally worn by infants and incontinent persons about the lower torso and having the general form of a sheet, different portions of which are fastened together to encircle the waist and the legs of the wearer.

The term “training pants” refers to an absorbent article generally worn by infants and incontinent persons about the lower torso and having the general form of a pair of short pants that can be applied or removed from the wearer without unfastening.

The term “refastenable” refers to the property of two elements being capable of releasable attachment, separation, and subsequent releasable reattachment without substantial permanent deformation or rupture.

The terms “releasably attached,” “releasably engaged”, and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment.

The term “toilet training” refers to the development of continence, which is the ability to voluntarily retain one's urine and feces. Individuals who are incontinent are unable to voluntarily retain their bodily discharges and, instead, urinate and defecate reflexively. For example, newborn babies are incontinent. Coincident with the development of continence, children typically develop the ability to voluntarily urinate and defecate, and cease reflexive elimination. This development of continence and of voluntary elimination, in place of reflexive elimination, may be accelerated and/or guided by caregivers through associative and conditioning techniques of training the child. For the purpose of the present disclosure, the term “toilet training” is used to denote training both for continence, itself, and for the voluntary elimination that is associated with continence. It is also noted that the term “toilet training” is synonymous with the term “potty training”.

The term “longitudinal” refers to a direction running parallel to the maximum linear dimension of the article and includes directions within ±45° of the longitudinal direction.

The term “lateral” or “transverse” refers to a direction running at a 90 degree angle to the longitudinal direction and includes directions within ±45° of the lateral direction.

The term “x-y plane” refers to the generally planar structure of a sheet material defined by its length and width and lies between the sheet material's two major surfaces regardless of whether or not the sheet material is flat or curved.

The term “z-direction” refers to the direction through the thickness of a sheet material and generally orthogonal to the x-y plane.

The term “attached” refers to elements being connected or united by fastening, adhering, bonding, etc. by any method suitable for the elements being attached together and their constituent materials. Many suitable methods for attaching elements together are well-known, including adhesive bonding, pressure bonding, thermal bonding, mechanical fastening, etc. Such attachment methods may be used to attach elements together over a particular area either continuously or intermittently.

The term “cohesive” refers to the property of a material that sticks to itself but does not to any significant degree stick to other materials.

The terms “proximal” and “distal” refer respectively to the location of an element relatively near to or far from the center of a structure, e.g., the proximal edge of a longitudinally extending element is located nearer to the longitudinal axis than the distal edge of the same element is located relative to the same longitudinal axis.

The terms “interior” and “exterior” refer respectively to the location of an element that is intended to be placed against or toward the body of a wearer when an absorbent article is worn and the location of an element that is intended to be placed against or toward any clothing that is worn over the absorbent article. Synonyms for “interior” and “exterior” include, respectively, “inner” and “outer”, as well as “inside” and “outside”. Also, when the absorbent article is oriented such that its interior faces upward, e.g., when it is laid out in preparation for setting the wearer on top of it, synonyms include “upper” and “lower” and “top” and “bottom”, respectively.

The terms “water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water to pass through its thickness in the absence of a forcing pressure. Conversely, the term “water-impermeable” refers to a layer or a layered structure through the thickness of which liquid water cannot pass in the absence of a forcing pressure. A layer or a layered structure that is water-impermeable according to this definition may be permeable to water vapor, i.e., may be “water vapor-permeable”. Such a water vapor-permeable layer or layered structure is commonly known in the art as “breathable”. As is well known in the art, a common method for measuring the permeability to water of the materials typically used in absorbent articles is a hydrostatic pressure test, also called a hydrostatic head test or simply a “hydrohead” test. Suitable well known compendial methods for hydrohead testing are approved by INDA (formerly the International Nonwovens and Disposables Association, now The Association of the Nonwoven Fabrics Industry) and EDANA (European Disposables And Nonwovens Association).

The term “retard” refers to the hindrance or the prevention of the flow of liquid water. In the context of the term “flow control layer”, both terms together refer to the fact that different layers in a layered structure may be water-permeable, yet differ in the respective flow rates at which they permit liquid water, and likewise bodily wastes that are aqueous in nature, to pass through their respective thicknesses. For example, a layer containing capillary channels and through whose thickness liquid water wicks in the absence of any forcing pressure is considered to be water-permeable. However, the flow rate at which liquid water can pass through the thickness of such a layer may be lower than the flow rate at which liquid water can pass through the thickness of a layer containing holes that are too large to act as capillary channels. Similarly, two layers both containing capillary channels and through whose thicknesses liquid water wicks in the absence of any forcing pressure are both considered to be water-permeable. However, the capillary channels in one of the layers may differ in size from those in the other layer or may be more numerous than those in the other layer, such that the wicking flow rate of liquid water through the one layer may be greater than that through the other layer. Thus, in a layered structure, one layer serving as a flow control layer may retard the passage of liquid water through the thickness of the layered structure, relative to the greater flow rate at which another of the layers would permit the passage of the liquid water through its thickness in the absence of the flow control layer. It is noted that when the flow control layer is water-impermeable, it effectively prevents the passage of liquid water through its thickness in the absence of a forcing pressure, i.e., the prevention of the passage of liquid water is included within the meaning of the term “retard”.

The term “visible” refers to the quality of being capable of being seen by the naked eye under conditions of normal room lighting or in natural light during the daytime. Becoming “more visible” or “less visible” means changing in visibility to a noticeable extent when viewed under a generally constant or equal lighting condition.

The term “visible highlighting” refers to the visible differentiation of an object such that it noticeably stands out from its surroundings, e.g., by differing in coloration, hue, or tint, by differing in lightness, darkness, or contrast, by differing due to the presence or absence of graphical or solid color forms, or by any other variation serving to create noticeable visible differentiation.

The term “coloring” refers to the effect produced by applying or combining colors in and/or on an object or a portion of an object.

The term “coloration” refers to the arrangement or degree of coloring especially when used to visibly differentiate an object or a portion of an object in order to visibly highlight it.

The term “solid coloring” refers to the unbroken, i.e., uninterrupted, coloring of an area as contrasted with the discrete line-like form of some graphics.

The term “graphic” refers to a product of graphic art or a graphic representation in a pictorial form. A graphic may be a symbol, shape, image, text, or other form of indicia.

The term “associative correlation” refers to establishing a mutual or reciprocal relation between the visible highlighting and that with which it is being associatively correlated so that an association, i.e. a mental connection or bond, is formed between the two. This term is used in the context of associatively correlating the respective visible forms of the visible highlighting and an externally visible graphics in or on the absorbent article as well as in the context of associatively correlating the visible highlighting or graphics with the concept of urinary toilet training, For example, associatively correlated graphics may serve in concert to draw attention to an opportunity for urinary toilet training when an absorbent article is viewed prior to its being worn, to provide an externally visible reminder of the presence of the wetness sensation member in the interior of the absorbent article while it is being worn, etc. Similarly, visible highlighting that provides a visual reference to a topic related to urinary toilet training, such as dryness, wetness, or protection from wetness, may serve to associatively correlate the visible highlighting to the concept of urinary toilet training and thereby facilitate an opportunity for urinary toilet training.

The terms “interactively interrelated”, “interactively unrelated”, “related in subject matter”, “unrelated in subject matter”, and “related by a common story line” are intended to have the same meanings as in U.S. Pat. No. 6,297,424 issued to Olson et al. on 2 Oct. 2001, U.S. Pat. No. 6,635,797 issued to Olson, et al. on 21 Oct. 2003, and U.S. Pat. No. 6,307,119 issued to Cammarota et al. on 23 Oct. 2001.

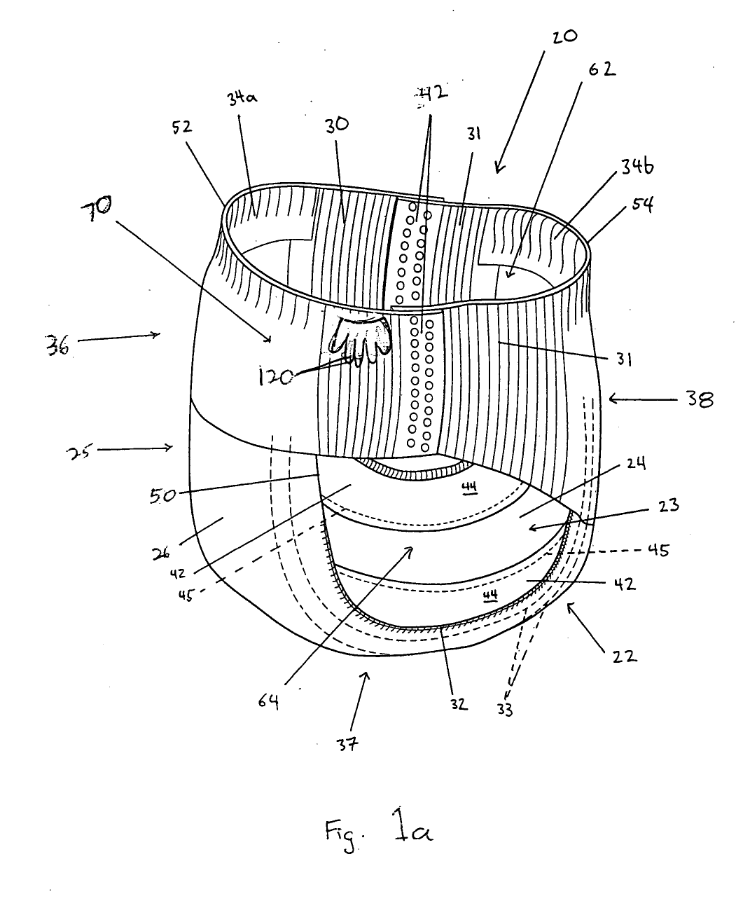

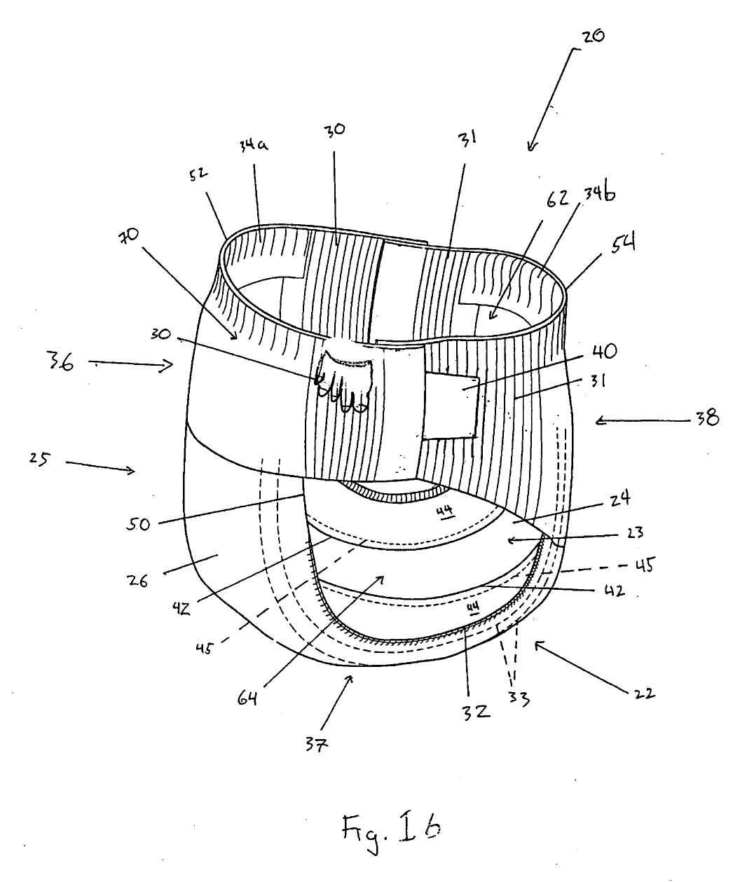

FIGS. 1a-b show a plan view of an exemplary disposable absorbent article in the form of a pull on type pant absorbent article 20. The absorbent article 20 may be configured with a front waist region 36 and a back waist region 38 opposed to the front waist region 36, and a crotch region 37 located between the front waist region 36 and the back waist region 38. The periphery of the absorbent article 20 is defined by the outer edges of the absorbent article 20 in which side edges 50 lie generally parallel to the longitudinal centerline 100 and the front waist edge 52 and back waist edge 54 lie generally parallel to the lateral centerline 110 of the absorbent article 20 and extend between the side edges 50.

The absorbent assembly 22 of the absorbent article 20 may include a liquid pervious topsheet 24, a liquid impervious backsheet 26, and an absorbent core 28 which may be positioned between at least a portion of the topsheet 24 and the backsheet 26. The absorbent assembly 22 may constitute the main structure of the diaper with other features added to form the composite diaper structure. The absorbent assembly 22 and generally all elements of absorbent article 20 may have a body-facing surface 23 which generally is in contact with the body or in close proximity to the body when the article is worn. The absorbent assembly 22 may have a garment-facing surface 25 opposed to the body-facing surface 23 and which generally contacts with or may be in close proximity to any garment being worn. The topsheet 24, the backsheet 26, and the absorbent core 28 may be assembled in a variety of configurations well known in the art. Exemplary absorbent assembly structures are described in U.S. Pat. No. 5,899,895 issued May 4, 1999 and U.S. Pat. No. 6,120,487 issued Sep. 19, 2000.

The backsheet 26 is generally that portion of the absorbent article 20 which is disposed adjacent the garment-facing surface of the absorbent core 28 and which prevents the excreta and/or exudates contained therein from soiling garments or other articles which may contact the absorbent article 20, such as bedsheets and clothing. In preferred embodiments, the backsheet 26 may be substantially impervious to liquid and may comprise any suitable thin plastic film known in the art, including a breathable film. Exemplars of suitable backsheet films include those manufactured by Tredegar Industries, Inc., or Terre Haute, Ind., USA, and sold under the trade names X15306, X10962, and X10964.

The backsheet 26 may be joined to the topsheet 24, the absorbent core 28 or any other element of the absorbent article 20 or absorbent assembly 22 by any attachment means known in the art. For example, the attachment means may include a uniform continuous layer of adhesive, a patterned layer of adhesive, or an array of separate lines, spirals, or spots of adhesive. Exemplars of suitable adhesives include those manufactured by H.B. Fuller Company of St. Paul, Minn., USA and marketed as HL-1620 and HL-1358-XZP. Alternatively, the attachment means may comprise heat bonds, pressure bonds, ultrasonic bonds, dynamic mechanical bonds, or any other suitable attachment means or combinations of attachment means known in the art.

The topsheet 24 is preferably disposed adjacent the body-facing surface of the absorbent core 28 and may be joined to the absorbent core 28 and/or to the backsheet 26 by any attachment means known in the art. The topsheet 24 is preferably compliant, soft-feeling, and non-irritating to the wearer's skin. Preferably, at least a portion of the topsheet 24 is liquid pervious, permitting liquids to readily penetrate through its thickness. A suitable topsheet may be manufactured from a wide range of materials known in the art, such as porous foams, reticulated foams, apertured plastic films, or woven or nonwoven materials of natural fibers such as wood or cotton fibers, or synthetic fibers such as polyester or polypropylene fibers, or a combination of natural and synthetic fibers. If the topsheet 24 includes fibers, the fibers may be spunbond, carded, wet-laid, meltblown, hydroentangled, or otherwise processed as is known in the art. One suitable topsheet material is a thermobonded carded web which is available as Supplier Code No. P-8 from Fiberweb North America, Inc., Simpsonville, S.C., U.S.A.

The absorbent core 28 may comprise any absorbent material which is generally compressible, conformable, non-irritating to the wearer's skin, and capable of absorbing and retaining liquids such as urine and other bodily exudates. The absorbent core 28 may be manufactured in a wide variety of sizes and shapes, for example, rectangular, hourglass, “T”-shaped, asymmetric, etc. The absorbent core 28 may include any of a wide variety of liquid-absorbent materials commonly used in disposable diapers and other absorbent articles, such as comminuted wood pulp, which is generally referred to as airfelt, cellulose wadding, meltblown polymers, chemically stiffened, modified, or cross-linked cellulosic fibers, tissue, absorbent foams including those prepared from polymerization of a high internal phase emulsion, superabsorbent polymers, absorbent gelling materials, or any other known absorbent material or combinations of materials. Exemplary absorbent core structures are described in U.S. Pat. No. 4,610,678 issued Sep. 9, 1986 and U.S. Pat. No. 5,260,345 issued Nov. 9, 1993.

Absorbent article 20 may include at least one leg cuff. FIGS. 1-2 show absorbent article 20 with two pairs of leg cuffs: gasketing cuffs 32 and barrier cuffs 42. Leg cuffs are known variously in the art as gasketing cuffs, containment flaps, “stand-up” elasticized flaps, barrier cuffs, leg bands, side flaps, and/or elastic cuffs. The leg cuffs may be constructed in any suitable configuration known in the art, including those described in U.S. Pat. No. 4,695,278 issued Sep. 22, 1987, and U.S. Pat. No. 4,795,454 issued Jan. 3, 1989.

The barrier cuff 42 may be formed by a flap 44 and an elastic member 45. The flap 44 may be a continuous extension of any of the existing materials or elements that forms absorbent article 20. For example, flap 44 may be a portion of the topsheet 24 treated to be hydrophobic or the flap 44 may be a discrete element separately attached to absorbent article 20. The elastic member 45 may be an elastic material that provides elasticity to the barrier cuff 42. It is desirable that elastic member 45 exhibits sufficient elasticity such that the barrier cuff may remain in contact with the wearer during normal wear thus enhancing the barrier properties of the barrier cuff 42. U.S. Pat. Nos. 4,808,178 and 4,909,803 describe disposable diapers having barrier cuffs that improve the containment at the leg regions.

The gasketing cuff 32 may be substantially inelastic or may be elastically extensible to dynamically fit at the wearer's leg. The gasketing cuff 32 may be formed by one or more elastic members 33 operatively joined to the topsheet 24, backsheet 26, flap 44, or any other substrate used in the formation of absorbent article 20. In one suitable embodiment, the gasketing cuff 32 has a plurality of elastic member 33 joined between the backsheet 26 and the flap 44. U.S. Pat. No. 3,860,003 describes a disposable diaper which provides a contractible leg opening having a side flap and one or more elastic members to provide an elasticized leg cuff.

In some embodiments, such as that shown in FIG. 1b, the absorbent article 20 may include a fastening system 40. The fastening system 40 preferably maintains the front waist region 36 and the back waist region 38 in a hoop configuration such that lateral force exerted by an elastic belt contributes to a circumferential tension when the absorbent article 20 is worn. The vector of the circumferential tension may be aligned substantially parallel to the front waist edge 52 and back waist edge 54, which form the waist opening 21. The fastening system may be disposed anywhere provided that the fastening system maintains the circumferential elastic belt during wear. The fastening system 40 may be disposed at least partially adjacent at least a portion of the side edges 50 of the front waist region 36 and/or the back waist region 38. In general, the fastening system 40 may comprise any known fastening means. For example, the fastening system 40 may comprise surface fasteners such as tape tabs, hook and loop fastening components, and/or hermaphroditic fastening components. Furthermore, the fastening system 40 may include buttons, hooks, buckles, and/or other fastening components. In some embodiments, the fastening system 40 may include refastenable fastening means that allow the absorbent article 20 to be opened and re-fastened, for ease of fitting on and removal from the body of the wearer and for adjustment while the absorbent article 20 is worn. In certain embodiments, the fastening system 40 may comprise an engaging member and a receiving member. Suitable combinations of engaging members and receiving members include, respectively, hook to loop; hook to hook; adhesive to substrate; selective adhesive to substrate; cohesive to cohesive; variant thereof; and combinations thereof. A suitable fastening system 40 is described in U.S. Pat. No. 5,242,436 issued Sep. 7, 1993.

In some embodiments, the absorbent article 20 may be provided in a pre-closed form as shown, for example, in FIGS. 1a-b. The pre-closed absorbent article 20 may have its opposing side edges 50 in the front waist region 36 and the back waist region 38 joined by seams 42. The seams 42 may be formed by any suitable bonding means known in the art which is appropriate for the specific materials employed. For example, suitable bonding means may include ultrasonic sealing, heat sealing, pressure bonding, adhesive bonding, sewing, autogenous bonding, and the like. The seams 42 may be permanent, that is, they may be bonded such that separation of the joined opposing side edges 50 requires the rupture or other destructive manipulation of the bonded materials that prevents refastening of the side edges 50.

The absorbent article 20 may alternatively have its opposing side edges 50 fastened together by any suitable fastening means, including those described above for the fastening system 40 as shown in FIG. 1b. In some embodiments, the fastening system 40 of a pre-closed absorbent article 20 may be refastenable such that absorbent article 20 can be opened and re-fastened. A refastenable fastener may provide for easier application, removal, and adjustment. In one embodiment of a pre-closed absorbent article 20 having a fastening system 40 as shown in FIG. 1b, the fastening system 40 may be disposed at least partially adjacent at least a portion of the side edges 50 of the front waist region 36 and/or the back waist region 38.

The absorbent article 20 of the present invention may include an elastic belt 70 such that, when absorbent article 20 is in a closed configuration, the front waist region 36 and the back waist region 38 are joined to yield a continuous waist edge comprising a front waist edge 52 and a back waist edge 54 which together define a waist opening 62 and two leg openings 64. The elastic belt 70 may be constructed from one or more elastic elements such that, in a closed configuration, a lateral force exerted by said elastic element contributes to a circumferential tension when the absorbent article 20 is extended or stretched for application or when the absorbent article 20 is worn. The elastic belt 70 may be formed by a variety of elements or combination of elements.

One suitable element that may be used to form the elastic belt 70 is a waist feature 34a, 34b. The waist feature 34a, 34b may be disposed longitudinally outwardly from at least one of the waist edges 56 of the absorbent core 28. The waist feature 34a, 34b may be disposed along the front waist edge 52 and/or the back waist edge 54 of the absorbent article 20; generally the waist feature 34a, 34b will form a portion of the front waist edge 52 and/or the back waist edge 54. The waist feature 34a, 34b may comprise one or more separate elements affixed to the absorbent article 20 and/or may comprise a continuous extension of another element or substrate of the absorbent article 20, such as the backsheet 26 and/or the topsheet 24. For example, as illustrated in FIGS. 1a-b, absorbent article 20 may have a front waist feature 34a and a back waist feature 34b that are discontinuous in relation to one another. Alternatively, the front waist feature 34a and the back waist feature 34b may overlap or be positioned proximate to one another so as to effectively perform as a single waist feature. Alternatively, the waist feature 34a, 34b may span a portion of both the front waist region 36 and the back waist region 38. It may be desirable for the waist feature to completely span the front waist region 36 and the back waist region 38 so as to provide 360° elasticity to the elastic belt.

The waist feature 34a, 34b may be at least laterally elastically extensible to provide circumferential tension at the diaper waist opening 62. The waist feature 34a, 34b may be constructed in any of several different configurations known in the art. In one embodiment, waist feature 34a, 34b may be a stretch laminate comprising one or more substrates with elastic members joined thereon or therebetween. An exemplary waist feature 34a, 34b may be a stretch laminate comprising two layers of nonwoven material with a plurality of elastic strands stretch bonded therebetween. Such a waist feature 34a, 34b may be formed discretely and then joined to the absorbent article 20 or the waist feature may be formed unitarily within the diaper. As an example of a unitary formation, the waist feature 34a, 34b may comprise a plurality of elastic strands stretch bonded between two existing layers or substrates of the diaper (e.g., between the topsheet and the backsheet). Other exemplary waist feature constructions include those described in U.S. Pat. No. 4,515,595 issued May 7, 1985 and U.S. Pat. No. 5,221,274 issued Jun. 22, 1993.

One suitable element that may be used to form the elastic belt 70 is one or more side panels 30, 31. The absorbent article 20 may also include side panels 30, 31 disposed in the in the front waist region 36 and the back waist region 38, respectively. The side panels 30, 31 may be constructed in any suitable configuration known in the art. The side panels 30, 31 may be elastically extensible. A suitable elastic side panel is described in U.S. Pat. No. 5,669,897 issued Sep. 23, 1997.

The side panels 30, 31 may be integral with the absorbent assembly 22 (i.e., they may be continuous extensions of one or more of the layers of the absorbent assembly 22) or they may be separately attached to the main absorbent assembly 22. Alternatively, the side panels 30, 31 may be made of multiple components or layers some of which are discrete (i.e., either attached separately to the main absorbent portion or separated therefrom by a gap) and some of which are continuous. An example of this type of construction is a diaper provided with an outer nonwoven cover which completely covers all areas of the absorbent article 20 including the side panels 30, 31 and the absorbent assembly 22.

The side panels 30, 31 together with the absorbent assembly may form pull-on absorbent article 20 having a waist opening and a pair of leg openings, when said pull-on diaper is in a closed configuration. As shown in FIGS. 1-2, the absorbent article 20 has a pair of front side panels 30 disposed generally transversely outward from the longitudinal edges of the absorbent assembly and at or near the front waist region 36. Similarly, the absorbent article 20 has a pair of rear side panels 31 disposed generally transversely outward from the longitudinal edges of the absorbent assembly and at or near the rear waist region 38. The respective waist regions 36, 38 together with the side panels 30, 31 may form a continuous waist opening when the side panels 30, 31 are joined such by the seam 42 in FIG. 1a or by the fastening system 40 in FIG. 1b. Similarly, the main absorbent assembly 22 and the side panels 30 also form leg openings.

The front side panels 30 and the rear side panels 31 may be joined by a bonding method to form a seam 42. The front side panels 30 and the rear side panels 31 may be bonded by any suitable bonding means known in the art which is appropriate for the specific materials employed. For example, suitable bonding methods may include ultrasonic sealing, heat sealing, pressure bonding, adhesive bonding, sewing, autogenous bonding, and the like. The seams 42 may be permanent in that separation of the joined side panels 30, 31 requires the rupture or other destructive manipulation of the side panels 30, 31 effectively preventing refastening of the side panels 30, 31.

As shown in FIGS. 1a-b, front side panel 30 and rear side panels 31 may be joined at a point such that each side panel 30, 31 had approximately the same lateral width. However, the side panels 30, 31 may be joined at various locations. Furthermore, while FIGS. 1a-b show a front side panel 30 and a rear side panel 31 being joined to form the waist opening 62 and a pair of leg openings 64, a single front or rear side panel may join the front waist region to the rear waist region thereby forming the waist opening and pair of leg openings.

In certain embodiments, it is desirable that the side panels 30, 31 be extensible and/or elastic. The side panels 30, 31 may be made extensible or elastic by any of a variety of techniques known in the art. For example, an elastic side panel 30, 31 can be made by disposing an elastic member, such as elastic strands or films, between facing layers of cover material, such as a non-woven material. Typically, in such a construction the elastic stands are attached to the facing layers while in a stretched configuration. After attachment, the strands are allowed to relax thereby gathering the facing layers and creating an elastic laminate. In an alternative method, elastic strands or a film can be attached to one or more facing layers in either a relaxed configuration or partially stretched configuration. The resulting laminate can be made stretchable (or more stretchable in the case of partially stretched strands or film) by subjecting the laminate to an elongation process which elongates the facing layers permanently, but the elastic stands or layer only temporarily. Such processes are known in the art as “zero strain” stretch laminate formation, and the elongation of such laminates may be accomplished with suitable means such as rollers, engaging teeth, or the like. Examples of zero strain activation processing and formations of resulting stretchable laminates are described in U.S. Pat. No. 5,167,897 issued to Weber et al. and U.S. Pat. No. 5,156,793 issued to Buell et al.

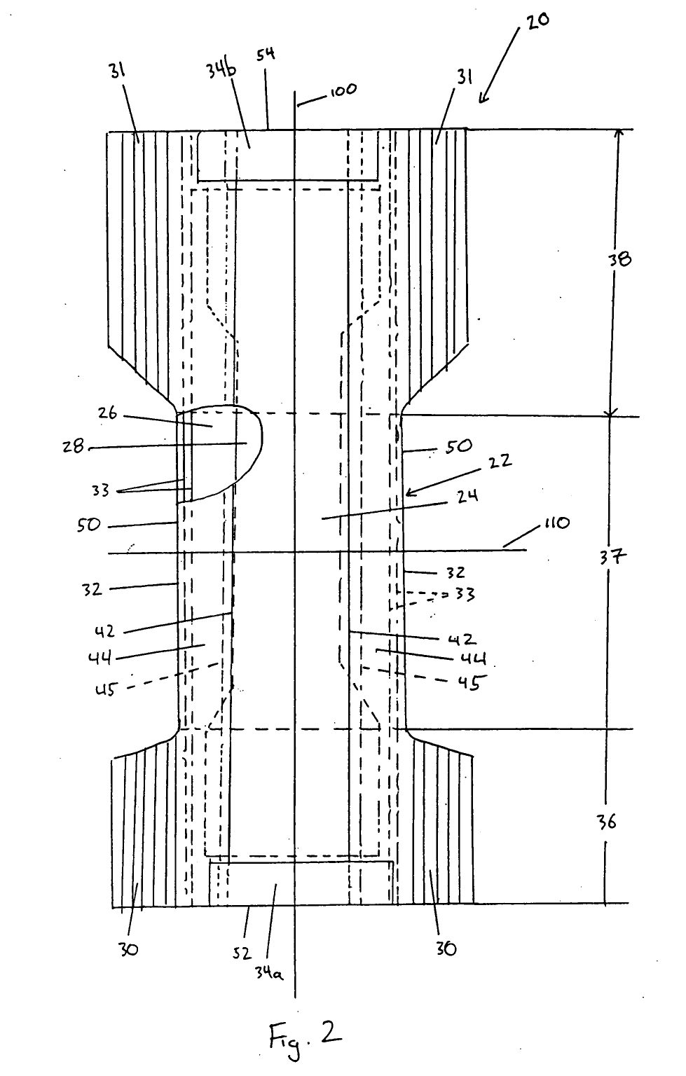

In certain embodiments, the elastic belt may be constructed from a combination of elements. For example, in FIGS. 1-2, the absorbent article 20 is shown with side panels and waist features. FIGS. 3 and 4 illustrates another embodiment of a pull-on diaper 320. FIG. 3 is a perspective view of diaper 320 having an absorbent assembly 322 and an elastic belt 370 in the form of a unitary waistband 380. The diaper 320 in open, unseamed form is shown in FIG. 4. Unless specifically stated otherwise, elements of the diaper 320 are substantially identical in composition and formation to the like elements in the embodiments provided above and shown in FIGS. 1-2.

The diaper 320 has a front waist region 336, a back waist region 338 opposed to the front waist region 336, and a crotch region 337 located between the front waist region 336 and the back waist region 38. The periphery of the diaper 320 is defined by the outer edges of the diaper 320 in which side edges 350 lie generally parallel to the longitudinal centerline 100 and the front waist edge 352 and back waist edge 354 lie generally parallel to the lateral centerline 110 of the diaper 320 and extend between the side edges 350.

The absorbent assembly 322 of the diaper 320 may include a liquid pervious topsheet 324, a liquid impervious backsheet 326, and an absorbent core 328 which may be positioned between at least a portion of the topsheet 324 and the backsheet 326. The absorbent assembly 322 may have an inner body-facing surface 323 which generally is in contact with the body or in close proximity to the body when the article is worn. The absorbent assembly 322 may also have an outer garment-facing surface 325 opposed to the inner surface 323 and which generally contacts with or may be in close proximity to any garment being worn. The topsheet 324, the backsheet 326, and the absorbent core 328 may be assembled in a variety of configurations well known in the art. Exemplary absorbent assembly structures are described in U.S. Pat. No. 5,899,895 issued May 4, 1999 and U.S. Pat. No. 6,120,487 issued Sep. 19, 2000.

Diaper 320 may include at least one leg cuff; FIGS. 3-4 show diaper 320 with two pairs of leg cuffs; gasketing cuff 332 and barrier cuff 342. Leg cuffs 332 are known variously in the art as gasketing cuffs, containment flaps, “stand-up” elasticized flaps, barrier cuffs, leg cuffs, leg bands, side flaps, barrier cuffs, and/or elastic cuffs. The leg cuffs may be constructed in any suitable configuration known in the art, including those described in U.S. Pat. No. 4,695,278 issued Sep. 22, 1987, and U.S. Pat. No. 4,795,454 issued Jan. 3, 1989. In FIGS. 3-4, the barrier cuff 342 is shown as being formed by a flap 344 and an elastic member 345, and the gasketing cuff 332 is shown with elastic members 333.

FIGS. 3-4 show the absorbent assembly operatively joined to a waistband 380. The absorbent assembly or any single element or subset of elements comprising the absorbent assembly may overlap the waistband 380. The absorbent assembly 322 is joined to the waistband 380 by any means known in the art including, but not limited to, ultrasonic sealing, heat sealing, pressure bonding, adhesive bonding, sewing, autogenous bonding, and the like.

The waistband 380 may encircle the waist opening 362 of the diaper 320. The waistband 380 may distribute and provide elastic resistance to the forces dynamically generated during wear. In a suitable embodiment as illustrated in FIGS. 3-4, waistband 380 may include a front waistband 380a and a back waistband 380b which may be joined by any means known in the art at a seam 342 to form a waist opening 362 and two leg openings 364. In this embodiment, the front waistband 380a and the back waistband 380b have an edge that corresponds to the front waist edge 352 and rear waist edge 354, respectively, of the diaper 320. The front waist edge 352 and rear waist edge 354 together define the waist opening 362. The absorbent assembly 322 may extend the entire longitudinal length of the front waistband 380a, the rear waistband 380b, or both. In certain embodiments, it is desirable that the absorbent core 328 does not extend into or overlap the waistband 380.

The waistband 380 may have an outer layer 392 and an inner layer 394. An elastic member 396 may be interposed between the outer layer 392 and the inner layer 394 to provide elasticity to the waistband 380. The front waistband 380a and the back waistband 380b may comprise the same materials and/or may have the same structure. Alternatively, the front waistband 380a and the back waistband 380b may comprise different materials and/or may have different structures. As shown in the embodiment of FIGS. 3-4, the front waistband 380a and the back waistband 380b generally have the same structure. While the outer layer 392 and the inner layer 394 are shown as being coextensive with the front and back waistband 380a, 380b, the outer layer 392 and the inner layer 394 may differ in size or orientation (e.g., the inner layer may be smaller than the size of the front and back waistband 380a, 380b). In a suitable embodiment, either the outer layer 392 or the inner layer 394 may extend beyond the other layer. The larger layer may be folded over and, optionally, bonded to form an edge for the waistband 380.

The waistband 380 may have any shape to provide a ring-like belt. In the embodiment shown in FIG. 3-4, the waist edge 352, 354 extends laterally straight and is substantially parallel to a lower waist edge 358. Alternatively, the waist edge 352, 354 and lower waist edge 358 may be shaped, curvilinear, and/or substantially nonparallel.

The waistband 380 may comprise a variety of suitable materials. Suitable material for the waistband 380 include a wide range of substrates such as plastic films; apertured plastic films; woven or nonwoven webs of natural materials (e.g., wood or cotton fibers), synthetic fibers (e.g., polyolefins, polyamides, polyester, polyethylene, or polypropylene fibers), or a combination of natural and/or synthetic fibers; or coated woven or nonwoven webs. The waistband 380 may comprise a stretchable nonwoven. In a suitable embodiment, the waistband 380 has an inner layer 394 comprising a hydrophobic, non-stretchable nonwoven material, an outer layer 392 comprising a hydrophobic, non-stretchable nonwoven material, and an elastic member 396 therebetween. Construction of elastic laminates is well known in the art. Construction may comprise the elastic member attached to the facing layers while in a stretched configuration. After attachment, the elastic member is allowed to relax thereby gathering the facing layers and creating an elastic laminate. Alternatively, elastic strands or film can be attached to one or more facing layers in either a relaxed configuration or partially stretched configuration. The resulting laminate can be made stretchable (or more stretchable as in the case of partially stretch strands or film) by subjecting the laminate to an elongation process which elongates the facing layers permanently, but the elastic stands or layer only temporarily. Such processes are known in the art as “zero strain” stretch laminate formation as described previously. In other embodiments, the waistband 380 may comprise the inner layer 394 and/or the outer layer 392 without an elastic member 396 if sufficient elasticity is present in the material which forms the inner layer 394 and/or outer layer 392 (e.g., layer may be an elastic scrim).

The elastic member 396 may comprise one or more of elastic elements such as strands or panels extending at least in the transverse direction. The elastic member 396 may be continuously or discontinuously disposed along the transverse width of the waistband. The elastic member 396 may be disposed evenly or disproportionately along the longitudinal length of the waistband 380. As shown in FIGS. 3-4, the elastic member 396 is in the form of strands continuously spanning the width of the waistband 380 and being substantially evenly spaced along the longitudinal length. It may be desirable that no elastic member 396 be provided in the portion of the waistband 380 which overlaps with the absorbent assembly 322; in such cases elastic member 396 may transversely span those portions of the waistband 380 that do not overlap the absorbent assembly 322.

Any of the application or removal aids or any of the sensory feedback features, described in greater detail below, may be provided on an absorbent article of any suitable basic configuration. Thus it should be understood that any or all such features can be provided on an absorbent article having the basic structure shown in FIGS. 1a or 1b, or shown in FIG. 3. Additionally, absorbent articles of other configurations (included taped diapers, and the like) can be provided with any or all of such features. For ease of description the application or removal aids and the sensory feedback features will be described with respect to an absorbent article of the type shown in FIGS. 1a or 1b.

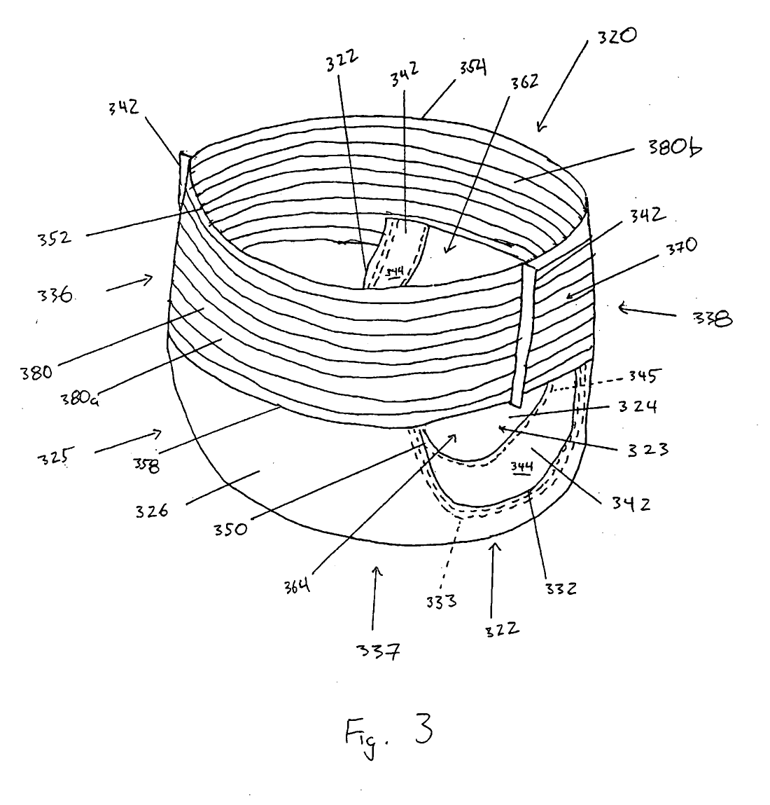

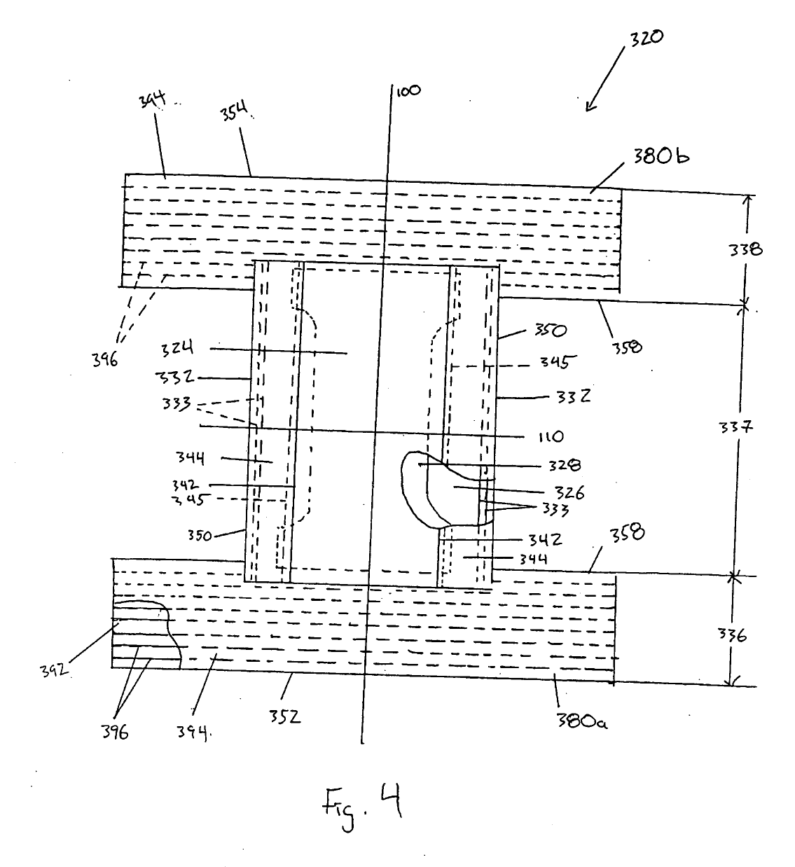

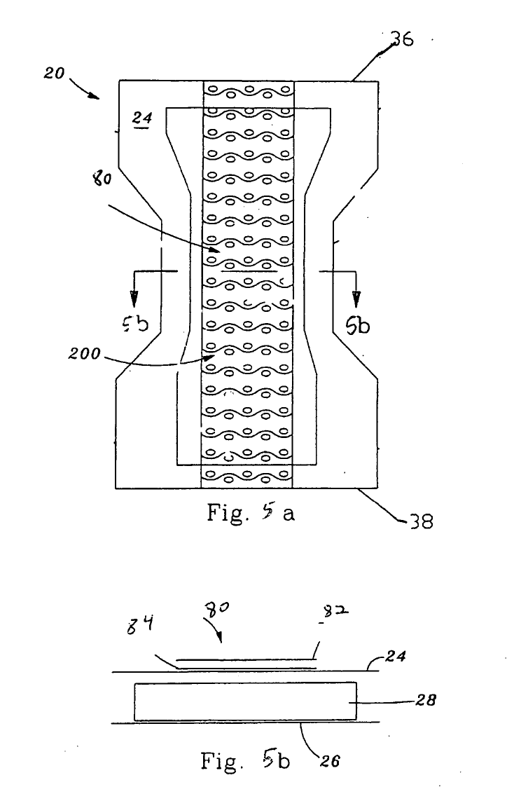

The absorbent article 20 may include a wetness sensation member. Several suitable structures for a wetness sensation member are described in U.S. Pat. No. 6,627,786 issued on 30 Sep. 2003 in the name of Roe et al. An exemplary wetness sensation member is shown in FIG. 5. The wetness sensation member 80 comprises a water-permeable body-facing layer (upper layer) 82 and a flow control layer 84 disposed in a face-to-face arrangement with the water-permeable layer 52. The flow control layer is preferably impermeable to liquid water but permeable to vapor so that it is breathable. Preferably, but not necessarily, some portion of the wetness sensation member and/or a layer to which the wetness sensation member is attached is configured to draw the wetness sensation member toward the skin of the wearer, such as by being elastically foreshortened, formed to have a lesser length than another layer disposed relatively exteriorly, etc.

During insults of urine, the water-permeable layer allows urine to penetrate in the z-direction and also provides a medium for the flow of urine in the x-y plane via wicking. The flow control layer retards the passage of the urine through the wetness sensation member in the z-direction, thereby expanding the wetted area of the wetness sensation member, which preferably is held in contact with the wearer's skin. The combination of limited penetration in the z-direction and wicking in the x-y plane causes the urine to spread out and effectively wet a large area before being absorbed into the absorbent assembly, thereby maximizing the wetness signal experienced by the wearer.

Exemplary water-permeable layers suitable for use in the wetness sensation members include nonwovens, foams, woven materials, etc. The water-permeable layer is preferably hydrophilic. Exemplary flow control layers suitable for use in the wetness sensation members include polyolefinic films, microporous or breathable films, other films, and hydrophobic nonwovens. Suitable hydrophobic nonwovens include SM (spunbond meltblown), SMS (spunbond meltblown spunbond), and SMMS (spunbond meltblown meltblown spunbond) composites.

The absorbent article 20 may include visible highlighting in the interior of the article to indicate the presence of the wetness sensation member or members and thereby facilitate an opportunity for the urinary toilet training of the wearer of the article. Such visible highlighting is described in co-pending and commonly assigned U.S. patent application Ser. No. 10/697,225 filed on 30 Oct. 2003 in the name of Davis et al. Although a wetness sensation member lacking this visible highlighting is fully functional in terms of providing a noticeable wetness signal to the wearer, the caregiver might overlook or forget the possibility of capitalizing on each opportunity for urinary toilet training if the body-facing portion of the absorbent article presents a generally uniform appearance, such as in absorbent articles that present a generally uniform white appearance on their body-facing surfaces.

Furthermore, once the caregiver decides to mention urinary toilet training to the wearer, the visible highlighting can serve to draw the wearer's interest or can be pointed out by the caregiver and incorporated into an explanation of the upcoming opportunity. Thus, the visible highlighting can provide a topic for conversation between the caregiver and the wearer on the subject of urinary toilet training and can likewise provide a nameable object for reference by the wearer, greatly simplifying the mental task required of the wearer who desires to communicate his or her need to go to the toilet or to communicate his or her improving recognition of the wetness signal provided by the wetness sensation member.

Even a simple solid coloring form of visible highlighting can serve to facilitate an opportunity for urinary toilet training, especially when used with wearers possessing some recognition of colors or colored forms. In addition, visible highlighting in the form of a color or colors may facilitate the teaching of recognition of colors and differences between colors, and the associated learning may enhance the urinary toilet training process in turn.

Because the wetness sensation member is located in what may be generally termed the laterally central region of the absorbent article, visibly highlighting the wetness sensation member may provide additional benefits related to the learning achieved by the wearer. For example, a visibly highlighted wetness sensation member may provide a line of reference for the visual separation of the two leg openings, including their differentiation into right and left leg openings for the respective feet to be inserted into the corresponding leg openings. Similarly, a longitudinally oriented visible highlighting may serve as a visual reference for the front to back direction, both for orienting the article prior to applying it, if done by the caregiver, or prior to donning it, if done by the wearer. This longitudinally oriented visual reference may also aid in the teaching of such skills as wiping one's self clean after using the toilet by using a longitudinal motion. The concept of something being central or “in the middle” may be taught and learned by visual reference to the visible highlighting and this concept may then be applied to related subjects, such as the anatomical location of the source of urine and the corresponding proper position in which to sit on the toilet. Thus, in the above and similar ways, the wearer can be made more aware of his or her own body, which may tend to enhance and facilitate the urinary toilet training experience.

In addition, the visible highlighting can serve to enhance the self-esteem of the wearer through a reminder that he or she is mature enough to be engaged in urinary toilet training. This effect can be compounded when the wearer succeeds in recognizing the need to go to the toilet and then sees the dry condition of the visibly highlighted wetness sensation member inside the article after pulling it down.

The visible highlighting may be provided by means of printing onto a surface of the wetness sensation member or one of its layers. For example, solid coloring or a graphic may be printed onto a surface of the flow control layer underlying the water-permeable layer. As another example, an adhesive or a gel may be printed onto a surface of either of the two layers. Such an adhesive or gel may be colored differently from the surrounding area. Alternatively, the adhesive or gel may be uncolored or may have the same color as the surrounding area, but may still provide visible highlighting by forming a distinctive raised area or pattern and/or by surrounding a distinctive recessed area or pattern.

The visible highlighting may also be provided by forming one or more layers of the wetness sensation member of a colored material, for example, a fibrous layer containing colored fibers, a monolithic layer containing a dispersed or imbedded colorant, a layer of an unbleached material that is colored in its virgin state, and so on.

In some embodiments, the visible highlighting may be provided by impressing or embossing the wetness sensation member or one of it layers. The impressed, embossed, or bonded portions of the wetness sensation member may provide a tactile sensation in addition to visibly highlighting the presence and location of the wetness sensation member. For instance, a raised area or a recessed area or the combination of raised and recessed areas adjacent to each other may be felt by the hand and, in some embodiments, may be felt by the wearer while wearing the article. Similarly, the raised area or pattern formed by a printed adhesive or gel, as mentioned above, may provide such a tactile sensation. Just as with the visible highlighting alone, the combination of visible highlighting and this tactile sensation can serve to draw the wearer's interest or can be pointed out by the caregiver and incorporated into an explanation of the upcoming opportunity for urinary toilet training.

In addition, the visible highlighting may be provided by incorporating distinctive fibers or filaments in one or both layers of the wetness sensation member or by distinctively orienting fibers or filaments in one of these layers. For example, a fiber or a filament of a distinctive color may be incorporated into the flow control material to visibly highlight its presence and its location in the article. Similarly, a distinctively thicker fiber or filament may be embedded in one of the two layers and thereby form a distinctive raised area or pattern.

If the portions of the structure of the absorbent article surrounding the wetness sensation member are of one color, the visible highlighting can be provided by the use of another color, by the use of contrast, by the use of a different pattern in the same or a similar color, or by any other method that visibly differentiates the wetness sensation member from the surrounding structural elements.

In some embodiments, the visible highlighting may include more than one color, more than one difference in contrast, more than one pattern, more than one graphic, more than one area of solid coloring, and so on, such that all portions of this description referring to the singular of a form of visible highlighting are meant to include the plural, and vice versa.

The visible highlighting may include open or closed geometric figures, a two dimensional representation of a three dimensional object, a representation of a commonly named or nameable shape or object, a representation of a recognizable object used in play, and/or a representation of a character that may be known to the wearer, such as a teddy bear, a character appearing on a television show for children, a character appearing in a game or a storybook for children, etc. In embodiments in which the visible highlighting includes a variety of figures, objects, and/or characters, the various elements of the visible highlighting may be interactively interrelated, related by subject matter, and/or related by a common story line. Conversely, the various elements may be interactively unrelated, unrelated by subject matter, and/or not related by a common story line.

When solid coloring is used, it may partially or completely fill the area bounded by a graphic outline, appear as shading inside or outside such a graphic outline, itself form a “filled-in” graphic, or simply uninterruptedly occupy an area, e.g., occupy the entire width of a layer of the wetness sensation member over all or a portion of the corresponding length.

In some embodiments, the visible highlighting may become more or less visible when the wetness sensation member is wetted. In addition, the visible highlighting may change color when the wetness sensation member is wetted. Any of these effects may be created by the use of inks or dyes or other agents that undergo chemical reactions or are dispersed or concentrated when wetted by urine. In general, any of the wetness indicating compositions commonly used in externally visible wetness indicators, such as so-called “appearing” or “disappearing” wetness indicators that may become more or less visible when wetted and in wetness indicators that may change color when wetted, may be used for these versions of visible highlighting.

It may be desired that rather than being structurally disposed in such a way as to provide a wetness indication that is visible from the outside of the absorbent article, any wetness indicating compositions used for the visible highlighting of the wetness sensation member may be visible from the body-facing surface of the absorbent article. This different disposition enables the caregiver to apply different techniques to the task of urinary toilet training when using an absorbent article of the present disclosure, as compared to using an absorbent article having only a wetness indicator visible from the outside of the article. For example, while the change in an exterior wetness indicator is visible for all to see, any change in the visible highlighting of an interior wetness sensation member remains “private” until either the caregiver or the wearer peers into the absorbent article or it is removed. Therefore, whether or not any wetting of the absorbent article has occurred can, itself, become the focus of a playful activity resembling a game, with the “secret” being revealed only when the caregiver and the wearer agree to conclude the game. If the wearer notices a sensation of wetness or merely desires to check the condition of the “private” indication, he or she can simply look inside the absorbent article. If the appearance of the visible highlighting has changed, the wearer can then choose to bring this to the attention of the caregiver in the context of asking to go to the bathroom. In addition, because the visible highlighting serves as a “private” indication, the wearer might be able to detect a change in its appearance before the appearance of any externally visible wetness indicator changes and thereby be the first person to mention the subject of going to the toilet. Furthermore, the provision of both visual and tactile sensations to the wearer may serve to reinforce the tactile sensation of wetness and thereby enhance the training effect of the wetness sensation member. An absorbent article in which the wetting is indicated by both a wetness sensation and a visible change in the appearance of the visible highlighting may thus facilitate faster learning on the part of the wearer.

Although the appearance of the visible highlighting remains “private” until either the caregiver or the wearer peers into the absorbent article or it is removed, the visible highlighting may be associatively correlated in visible form with marking that is located elsewhere in or on the absorbent article and is visible from the outside of the absorbent article. This externally visible marking may be permanent or may change in appearance while the absorbent article is being worn. For example, the externally visible marking may be an externally visible wetness indicator. By giving the visible highlighting of the wetness sensation member a visible form that is similar to the visible form of an externally visible marking, an opportunity for urinary toilet training may be enhanced. For instance, the caregiver can point out the similarity between the externally visible marking and the “private” visible highlighting of the wetness sensation member and ask the wearer to remember the hidden visible highlighting every time he or she notices the externally visible marking.

Even in embodiments in which the appearance of the visible highlighting is not affected by its being wetted, the associative correlation of the respective visible forms of an externally visible marking and the visible highlighting may serve to facilitate an opportunity for urinary toilet training. For example, if both the externally visible marking and the visible highlighting have the visible form of similar graphics, the externally visible marking can serve to draw the wearer's interest or can be pointed out by the caregiver and incorporated into an explanation of the ongoing opportunity for urinary toilet training.

Such associative correlation of the respective visible forms of an externally visible marking and the visible highlighting can be achieved without the respective visible forms being similar, so long as the respective visible forms are mutually related in a recognizable way. For example, the visible forms may be related in subject matter and/or may be related by a common story line and/or be interactively interrelated. Even an associative correlation of a simple solid coloring form of an externally visible marking with a similar solid coloring form of visible highlighting can serve to facilitate an opportunity for urinary toilet training, especially when used with wearers possessing some recognition of colors or colored forms.

Alternatively, the visible highlighting may be associatively uncorrelated with any externally visible marking. The lack of associative correlation may be complete or may be specific, e.g., the respective visible forms of the visible highlighting and the externally visible marking may be unrelated in subject matter, not related by a common story line, and/or interactively unrelated, while still being associatively correlated in another way.

The visible form of the visible highlighting of the wetness sensation member need not be associatively correlated with the concept of urinary toilet training. However, in some embodiments, the visible form of the visible highlighting may be associatively correlated with the concept of urinary toilet training by, for example, providing a visual reference to the liquid-related nature of urinary toilet training, such as wetness, dryness, protection from wetness, the flow of a liquid, water, et cetera, and thus may serve to facilitate an opportunity for urinary toilet training.

The visible highlighting may emphasize dryness by depicting the sun, fair weather clouds, a sunny day, etc., while wetness may be referenced by a depiction of a water puddle, a cloud with falling rain, etc. A visual reference to protection from wetness may be provided by a depiction of an umbrella, a raincoat, a rain hat, galoshes, a submarine, or some other object that may be associated by the wearer with the concept of staying dry in a wet environment.

In any of these visible forms of visible highlighting that are associatively correlated with the concept of urinary toilet training, a human form and/or a recognizable character may be depicted in the visible highlighting. For example, a child may be shown in conjunction with inanimate objects, a child may be shown sitting on a potty chair, and/or a character from a children's storybook or a children's television program may be shown in similar poses, etc.

The wetness sensation member according to the present disclosure may be arranged in an absorbent article in a variety of configurations. In addition, absorbent articles may include a single wetness sensation member or a plurality of wetness sensation members. In any event, the wetness sensation member(s) are preferably a part of, or attached to, an element or web, such as a topsheet, which is reliably held against the skin of the wearer. The wetness sensation member may extend over a portion of the disposable absorbent article spanning less than one half of the length of the article or else extend over a substantial part of the article spanning more than one half the length of the article. In addition, the wetness sensation member(s) are preferably positioned within the absorbent article to enhance the likelihood of being wetted with urine.

The wetness sensation member may also be releasably attached to or releasably engaged with the remainder of the absorbent article. In such a configuration, the wetness sensation member may be optionally removed from the absorbent article if the wetness sensation functionality is not desired. Such releasable attachment may be accomplished by a variety of known attachment means including adhesives, cohesives, ultrasonic bonding, thermal bonding, mechanical fasteners, or the like. Additionally, a removable wetness sensation member such as that described above may be provided with instructional graphics, printing, or the like indicating the fact that such a member may be removed and how such removal may be accomplished.

An exemplary embodiment of a absorbent article 20 including a wetness sensation member 80 disposed with the topsheet 24 is illustrated in FIG. 5a and FIG. 5b. The wetness sensation member in this embodiment is a separate composite member attached to the topsheet. The wetness sensation member comprises a water-permeable body-facing layer 82 and a flow control layer 84 disposed in a face-to-face arrangement with the body-facing layer. The visible highlighting 200 is shown in FIG. 5a as an exemplary pattern of wavy lines and circles.

The wetness sensation member 80 may have elastic properties and includes a first longitudinal end attached to the first waist region and a second longitudinal end attached to the second waist region. In addition, a center portion of the member may be attached to the crotch region in order to stabilize the member and facilitate fitting the article to the wearer, prevent interference with bowel movements and ensure good contact with the wearer's skin.

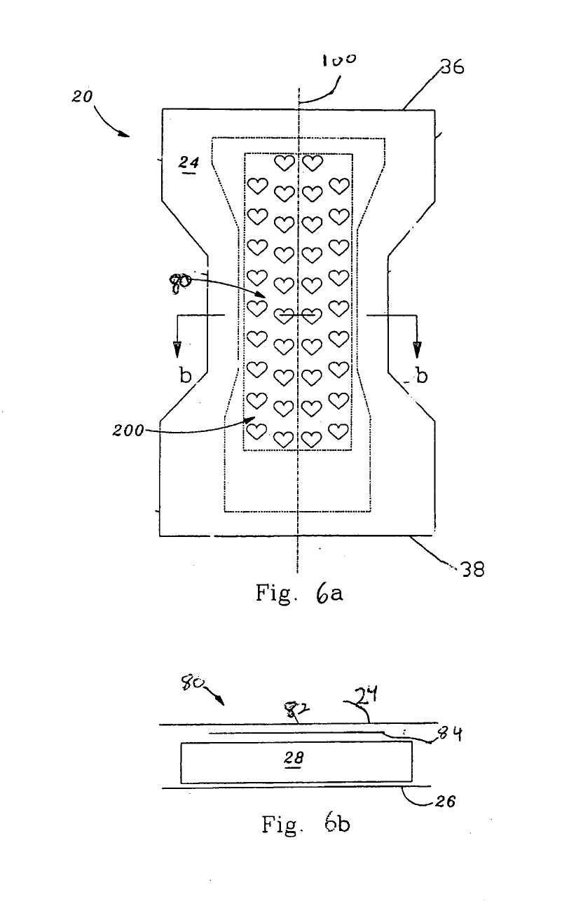

In an alternate embodiment shown in FIG. 6a and FIG. 6b, the flow control layer 84 of the wetness sensation member 80 is attached to the inner surface of the topsheet 24 such that a portion of the topsheet 24 serves as the water-permeable layer 82 of the wetness sensation member 80. For this embodiment, the topsheet 24 is preferably elastically foreshortened to deflect the wetness sensation member 80 into contact with the wearer's skin. Alternatively, this embodiment may include a topsheet that is shorter in length than the backsheet, having the longitudinal ends of the topsheet contiguous with the longitudinal ends of the backsheet so that as the disposable absorbent article is fitted around the wearer, the topsheet is forced into contact with the wearer's skin. The visible highlighting 200 is shown in FIG. 6a as an exemplary pattern of heart shapes.

Absorbent articles according to the present disclosure may include a plurality of wetness sensation members disposed on the body-facing surface of the article. For example, two flow control layers may be attached to the bottom surface of the topsheet 24 forming two wetness sensation members. For this embodiment, the flow control layers are disposed between the topsheet and the absorbent assembly 22 so that the topsheet serves as the water-permeable layers 82 of the wetness sensation members. The two flow control layers may be disposed parallel to and spaced apart from the longitudinal centerline 100 of the absorbent article 20. The spacing is determined to allow enough liquid to pass through to the core so as to prevent flooding that can result in leakage of the absorbent article during urination, while at the same time allowing enough liquid to flow and wick toward the flow control layers forming the wetness sensation members. The spacing between the flow control layers can be about 10 mm but can range from about 5 mm to about 15 mm and from about 8 mm and to about 12 mm. Although the embodiment described here has only two wetness sensation members, other absorbent article embodiments having three or more wetness sensation members are contemplated.

In another alternate embodiment the flow control layers may be disposed in two parallel oppositely facing Z-folds formed in the topsheet 24 along the longitudinal length of the diaper thus forming two wetness sensation members. The Z-folded topsheet may be attached to the underlying layers along the longitudinal edges of the topsheet 24, thus allowing the portion of the topsheet between the Z-folds to float freely. Longitudinally extending elastic elements may be disposed along the flow control layers in order to deflect the center portion of the Z-folded topsheet away from the absorbent assembly 22. As an alternative to a portion of the topsheet serving as a layer of a wetness sensation member, other components of the disposable absorbent article such as the barrier leg cuffs may serve as such a layer. The barrier leg cuffs may be made from either water-permeable or water-impermeable material. In either case, the barrier leg cuff material may serve as one of the layers of the wetness sensation member. In such exemplary embodiments, the structure of the barrier leg cuffs preferably holds the wetness sensation members in contact with the skin of the wearer to provide the sensation of wetness against the wearer's legs and/or crotch crease.

The embodiments of wetness sensation members disclosed hereunder perform most effectively when held in contact with the skin of the wearer. In order to ensure that contact is made with the wearer's skin during use, the body-facing portion of the wetness sensation members may include a body-adhering composition, such as a topical adhesive, which acts to hold the wetness sensation member in place during use. The body-adhering composition may be applied to at least a portion of the body-facing surface of the wetness sensation member. However, the body-adhering composition may also be integral with the material making up the body-facing layer of the wetness sensation member. Further, the body-adhering composition may be disposed on any portion of the wetness sensation member contacting the skin of the wearer in any pattern or configuration including, but not limited to lines, stripes, dots, and the like. Such a body-adhering composition may include any of one or more substances capable of releasable adhering to the skin of the wearer, such as those disclosed in U.S. Pat. Nos. 4,231,369, 4,593,053, 4,699,146, 4,738,257, 5,726,250, 4,078,568, 4,140,115, 4,192,785, 4,393,080, 4,505,976, 4,551,490, 4,768,503, 5,614,586, and 5,674,275, and in the PCT Application published as WO 94/13235A1.

As previously mentioned, the exemplary disposable absorbent article 20 includes refastenable side seams that can be used to fasten the waist regions together at the sides to apply the article like a diaper onto the body of the wearer and that can also be used to configure the article like a pair of pull-on training pants. The refastenable side seams can be fastened by the user before the article is applied onto the body of the wearer and the article can then be applied like a pair of pull-on training pants. The refastenable side seams can be opened and refastened after the article is applied onto the body of the wearer in order to gain access for the inspection of the interior of the article and/or to adjust its fit while being worn. Of course, the side seams can also be opened for the ultimate removal of the article for disposal, as an alternative to leaving the article in the form of a pair of training pants and pulling it downward over the legs and feet for removal. The refastenable side seams also facilitate the pre-configuration of the article in the form of a pair of training pants prior to the point of sale to the consumer, if such a pre-fastened presentation is desired by the manufacturer, distributor, and/or retailer, while still providing the user with the alternative of opening the side seams in preparation for applying the article like a diaper.

The primary fastening component may be formed of any material and in any form that will releasably attach to the mating surface of the opposing waist region when pressed against it. For example, the primary fastening component may be a mechanical fastener that releasably engages with the mating surface, such as by means of a plurality of hooks engaging with loops formed by fibers in a nonwoven sheet. Alternatively, the primary fastening component may be an adhesive that releasably adheres to the mating surface.

As described in more detail below, the primary fastening component may also interact with a discrete mating fastening component. For example, a mechanical primary fastening component containing hooks may engage with a discrete mating fastening component containing loops. Similarly, an adhesive primary fastening component may adhere to a discrete mating fastening sheet that is specifically selected for good adhesion. Also similarly, a cohesive primary fastening component may cohere to a mating cohesive fastening component. Each of the fastening components may have any suitable shape, such as rectangular, circular, ovoid, undulating, etc. The shape may be chosen according to various criteria, such as to maximize or minimize the area of the fastening component, to impart a particular appearance to the fastening component, to distribute the stresses and forces to which the fastening component is subjected when the article is worn in a particular way, etc.

Another example of refastenable side seams may include primary fastening components disposed on the exterior of the diaper in one waist region and discrete mating fastening components correspondingly disposed on the interior of the diaper in the other waist region. In addition, secondary fastening components may be disposed laterally adjacent to the primary fastening components.

When both primary fastening components and discrete mating fastening components are present, their disposition relative to the interior and exterior of the disposable absorbent article is generally interchangeable.

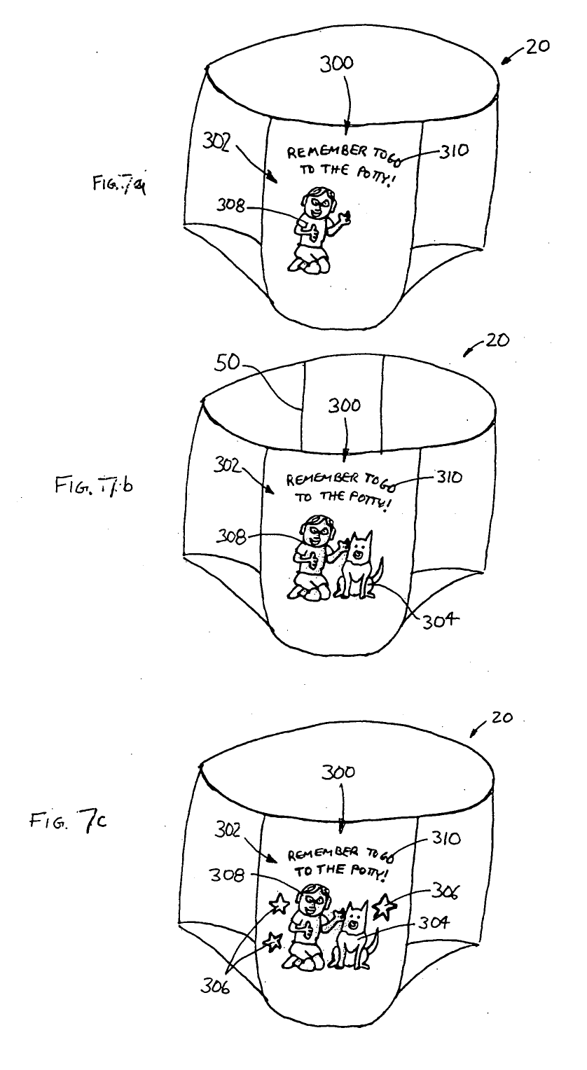

The absorbent article 20 may further include graphics 300 which facilitate toilet training, as illustrated in FIGS. 7a-c. In the exemplary embodiment, the graphics 300 include a permanent graphic 302, a first appearing graphic 304, and a second appearing graphic 306. The permanent graphic 300 may include a character image 308 resembling a boy and a text graphic 310 including words forming a message, such as “Remember to go to the potty!” As illustrated, the boy in the character image 308 is kneeling on the ground and the text graphic 310 is located above the character image 308. While the permanent graphic 302 is illustrated as including the character image 308, it will be appreciated that the permanent graphic 302 may include other graphics such as an object, design, or pattern. Furthermore, character images other than a boy may be provided, such as a girl, an animal (which may be anthropomorphic), a cartoon character, and the like. Still further, additional or alternative text may be provided in the permanent graphic 300.

The first appearing graphic 304 is illustrated as a character image that may be associatively correlated to the permanent graphic 302. In the illustrated embodiment, the first appearing graphic 304 is in the form of a dog sitting next to the boy character image 308, with the boy character image 308 appearing to pet the dog. As with the permanent graphic 302, the first appearing graphic 304 may be in the form of a different character other than the dog, or may be in the form of something other than a character, such as an object, design, pattern, background color, or text.

The second appearing graphic 306 is illustrated as an object image that may be associatively correlated to the permanent graphic 302 and the first appearing graphic 304. In the illustrated embodiment, the second appearing graphic 306 is in the form of a plurality of stars located proximate the permanent graphic 302 and the first appearing graphic 304. The second appearing graphic 306 may be in the form of a different object other than a plurality of stars, or may be in the form of something other than an object, such as a character, design, pattern, background color, or text. Still further, while the exemplary embodiment shows three stars, fewer or less than three objects may be provided as the second appearing graphic 306.

As noted above, the permanent, first appearing, and second appearing graphics 302, 304, 306 may be associatively correlated to one another to form a scene. Accordingly, the graphics may relate to a common theme or story line. While the illustrated embodiment shows a scene including a boy, dog, and star images, other scenes may be provided. For example, the scene may include images of a girl, a flower, and a rainbow, or a cat, a cow, and a moon, as but two examples. Similarly, the graphics may include images which children already associate with each other, such as a cartoon or popular entertainment character and the typical friends, partners, or objects that appear with the main character. When such familiar images are used, the child will expect the second character or item that is usually associated with the partial scene to appear, thereby maintaining the child's interest and encouraging him or her to complete a toilet training task, such as staying dry, until the scene is complete.

Alternatively or additionally, the graphics may be associatively correlated based on their proximity to one another. The graphics may include multiple separate images that form a complete scene having multiple interrelated objects or characters as noted above. Alternatively, the multiple graphics may build a unitary final image. In this case, for example, the permanent image may be of an object or character that is intrinsically or inherently incomplete, such as a partial drawing like a flower stem without a flower. The appearing graphics may be images of additional parts of the flower, such as flower petals, leaves, and the like. Accordingly, the child will remain interested in the toilet training task at least until completion of the final, composite image.

The graphics may be in the form of any visual representation that attracts the attention of, or is otherwise identifiable by, the wearer. The graphics may include one or more icons, which may comprise, but are not limited to, pictorial symbols, photographs, drawings, cartoons, and logos. For example, the icons may be provided as drawings of a child or an anthropomorphic image of an animal using the absorbent article 20. Similarly, the icons may include well-known cartoon characters or brand logos, or characters specifically created to be associated with the article. The icons may further include symbols, such as arrows, to indicate motion, movement, or directionality.

The graphics may be arranged in any manner as long as they are viewable by the wearer. The graphics may include a single icon or a series of icons. If a series of icons is provided, each icon may be different. Different icons may be complementary to one another, in that they are related to the same concept or activity, or incorporate a common visual element (such as a similar appearance, color, or theme). The icons may be arranged in any suitable fashion, such as, but not limited to, vertically, horizontally, diagonally, circular, arcs, and combinations thereof.

The graphics may optionally include a character image that can increase a user's interest in the product. The term “character image” is used herein to refer to a graphic containing an anthropomorphic image, and in particular an image having or suggesting human form or appearance which ascribes human motivations, characteristics or behavior to inanimate objects, animals, natural phenomena, toys, cartoon characters, or the like. The character image may be associated with popular characters in the media, advertising or well known in a particular culture. Ideally they are characters that the user, particularly if a child, cares about and wants to identify with.

The graphics disclosed herein are particularly suited for absorbent articles intended for use by children. Accordingly, the exemplary images illustrated herein may be cognitively functional to a pre-literate child. While the graphics may include text, the primary form of communication may be symbols, icons, or other markings other than words, so that a pre-literate child may comprehend and follow the instructions or other information indicated by the graphics.

As used herein, the terms “pre-literate” and “incapable of reading” are used interchangeably to mean the inability of a child to correctly understand, comprehend and follow prompts written in a language that the child can speak without assistance of a caregiver. The ability of a child to recognize letters and/or read one or two isolated words still means that the child is “incapable of reading” since he or she is unable to understand, comprehend and follow such written prompts, without assistance. However, this definition of “incapable of reading” does not exclude the child from being able to understand, comprehend and follow visual prompts which are presented in the form of drawings, icons, symbols, gestures, cartoons and the like. Furthermore, while the disclosed embodiments are capable of being understood by a pre-literate child, it is not necessary for the images to be understood at this level.

The first and second appearing graphics 304, 306 may appear at different time intervals to build interest and encourage the child not to urinate in the article 20. Use of appearing graphics allows a caregiver to explain the appearance of a new graphic for a reward, and therefore a more positive approach to toilet training may be taken. For example, the appearing graphic may be viewed as a reward for the child staying dry. Accordingly, each of the first and second appearing graphics 304, 306 has an initial state, in which the graphic is transparent, translucent, or relatively less visible, and a subsequent state, in which the graphic is at least semi-opaque or otherwise relatively more visible. The first appearing graphic 304 may change from the initial state to the subsequent over a first time period, such as, for example, approximately one hour. The second appearing graphic 306 changes from the initial state to the subsequent state over a second time period that may be different from the first time period, such as approximately two hours. Accordingly, the child is initially encouraged by the appearance of the first appearing graphic 304 and learns to anticipate and appreciate the appearance of the second appearing graphic 306 at a later time.

The first and second appearing graphics 304, 306 may become visible at either a uniform or a variable rate. For example, the graphics may appear slowly or gradually over time, such as substantially at the same rate over a period of time such as approximately one hour or approximately two hours. Alternatively, the graphics may appear at a variable or non-uniform rate. For example, the graphics may show no change in visibility for an initial period of time followed by a period of more rapid change in visibility. As but one example, the graphics may remain substantially hidden, obscured, or less visible for a period of approximately 50 minutes and then more rapidly change to a more visible state over a period of approximately 10 minutes. Furthermore, while the exemplary second time period of two hours is twice the exemplary first time period of one hour, the time periods need not be related by any relative ratio. Instead, the time periods may include any suitable time interval. In addition, while the exemplary embodiment illustrates first and second appearing graphics 304, 306, a third or more appearing graphics may be provided having different time periods for changing from the initial to the subsequent state without departing from the scope of this disclosure.

The appearing graphics 304, 306 may become less visible when subjected to liquid such as urine, thereby to discourage a child from urinating in the absorbent article 20. Accordingly, the appearing graphics 304, 306 may be positioned or otherwise placed in liquid communication with the absorbent assembly 22, meaning that liquid such as urine is capable of moving between the appearing graphics 304, 306 and the absorbent assembly 22 under ordinary use conditions. Consequently, when a child wets the absorbent article 20, liquid is communicated to the appearing graphics 304, 306, whereupon the appearing graphics dissolve, change color, disappear, or the like. For example, should the first appearing graphic 304 as shown in FIG. 7b, or the first and second appearing graphics 304, 306 as shown in FIG. 7c, be in the subsequent state and hence visible, the appearing graphics 304, 306 will disappear upon contact with urine, leaving the permanent graphic 302 as shown in FIG. 7a.

The permanent graphic 302, as well as the first and second appearing graphics 304, 306 when in the subsequent state, are viewable from an exterior of the absorbent article 20. Specifically, the graphics are viewable at the exterior surface of the backsheet 26. Accordingly, the graphics may be disposed on the backsheet 26, the absorbent assembly 22, or a layer located therebetween. For purposes of this disclosure, should a separate, intermediate layer of material be located between the backsheet 26 and the absorbent assembly 22, the intermediate layer will be considered to be associated with at least one of the backsheet 26 and the absorbent assembly 22, and therefore a graphic disposed on such an intermediate layer is considered to be disposed on at least one of the backsheet 26 and the absorbent assembly 22.

Various placements of the graphics 302, 304, 306 may be better understood with reference to the partial section views of absorbent articles that are shown in FIG. 8a and FIG. 8b. The various layers of the illustrated embodiments can be secured together using adhesives, thermal bonds, mechanical bonds, or other means known to those skilled in the art.

FIG. 8a illustrates a partial section view of an absorbent article having an absorbent core 28 sandwiched between a backsheet 26 and a topsheet 24. The illustrated backsheet 26 consists of a single layer having an exterior surface 320 and an opposite interior surface 322. The permanent and appearing graphics 302, 304, 306 may be disposed on the backsheet 26, which includes on either surface 320 or 322 of the backsheet, on an exterior surface 324 of the absorbent core 28, or between the absorbent core and the backsheet. The backsheet 26 is preferably formed of a material that is liquid impermeable. The permanent graphic 302 and appearing graphics 304, 306 need not be located in the same position or on the same substrate.

FIG. 8b illustrates a partial section view of another absorbent article having an absorbent core 28 sandwiched between a backsheet 26 and a topsheet 24. The illustrated backsheet 26 consists of a two-layer composite comprising an outer layer 326 and an inner layer 328. The backsheet 26 has an exterior surface 320 and an opposite interior surface 322. The permanent and appearing graphics 302, 304, 306 may be disposed on the backsheet 26, which includes, in particular, on the exterior surface 320, on the interior surface 322, between the outer and inner layers 326, 328, on either or both facing surfaces of the outer and inner layers 326, 328, on the exterior surface 324 of the absorbent assembly 28, or between the absorbent assembly and backsheet.

Various types of mechanisms may be used to obtain the desired time periods between the initial and subsequent states of the appearing graphics 304, 306. For example, the graphics 304, 306, may be formed by a chemical composition that exhibits different characteristics, such as different colors, when subjected to altered environmental conditions. A dye, such as methylene blue, may be used which is colorless when in a reduced state but which turns blue in an oxidized state. To reach the oxidized state, the dye must be exposed to oxygen. Accordingly, if the appearing graphics 304, 306 are formed of methylene blue, they will initially be colorless but subsequently turn blue (and visible) when oxidized by sufficient exposure to atmosphere. Initiation of the oxidation may be controlled by sealing the appearing graphics 304, 306 prior to use, such as by covering with a membrane or plastic that is removed approximately at the time the article is first worn. The methylene blue dye has the added benefit of being liquid soluble, and therefore may be flushed to the absorbent core 28 or other area of the article when exposed to urine, thereby causing the appearing graphics 304, 306 to disappear, become obscured, or otherwise be less visible.

Alternatives to the foregoing dye composition may be used to effect an appearing graphic. Inks or dyes that change appearance when subjected to different temperatures, conductivity or resistivity, or other surrounding conditions may be used. Still further, rather than a special ink or dye, a mechanical structure may be provided that controls flow of ink from an obscured location to a location viewable from the exterior of the article may be used. The ink or dye may be stored in a reservoir that is at least initially not viewable from an exterior of the article. A filter, capillary tube, or other mechanical structure may control flow of the dye to an exteriorly visible location, effectively creating a time release of the dye. The appearing graphic may use electrical means to measure time periods and/or release ink or other graphic material. For example, a simple circuit for measuring time may be provided, thereby equipping the article with an electronic timer that may be powered by a small battery or other power source. The timer circuit may generate an electronic signal indicating the desired time for releasing ink or the like from a storage location.

The absorbent article 20 may include structure for providing feedback to a child using multiple senses, thereby more clearly indicating the desired behavior during toilet training. The article 20, for example, may include at least the first appearing graphic 304 to provide positive encouragement to stay dry and also at least wetness sensation member, such as member 50, to provide negative reinforcement or otherwise inform the child or caregiver that an accident has occurred. The appearing graphic 304 generates a visual cue received by the sense of sight, while the wetness sensation member 50 provides a tactile signal received by the child's sense of touch. By providing feedback receivable by multiple senses, the child is more apt to learn and remember the desired behavior for toilet training.

In addition to the appearing graphics described above, the absorbent article 20 may be provided with disappearing graphics. Such disappearing graphics may disappear with dryness (as for example, as the result of oxidation) or with wetness (as for example by dissolving inks). Additionally, appearing graphics may be used which appear with wetness. Both dryness and wetness appearing or disappearing graphics may be used in combination in the same article.

As previously noted, it may be desired to provide the multi-functional training garment such as absorbent article 20 with features allowing for ease of application (donning) and/or removal. Such features may include a thin core structure (e.g. one without undue or excess bulk) allowing the absorbent article 20 to be pulled up easily between the wearer's legs. Other features, which are described in greater detail below may include informational images and slow recovery portions of the absorbent article 20. As with the training features described above, these features may be used alone or in any desired combination with the remaining features described.

As is shown in FIG. 1a, an informational image 120 may be disposed on at least one of the side regions (such as side panel 30) to communicate information relating to the pull-up absorbent article 20 to a user. The informational image 120 is selected to communicate information regarding the use of absorbent article 20 to a user. The information may relate to a characteristic or feature of the absorbent article 20, or may provide instructional or descriptive information regarding the use of article 20.

As used herein, the term “communicate” refers to the ability of the informational image to impress an idea or message upon, or trigger a cognitive response within, a user. As such, communication may rely upon a user's experience or knowledge to arrive at the intended message. Additionally or alternatively, the image preferably illustrates simple concepts that are understood at a basic or visceral level that does not require the prior knowledge or experience of a user. In any event, the image is preferably cognitively functional in that it conveys a message, preferably related to an action or decision to be made, that is generally capable of being understood by a recipient user.

As used herein, the phrase “disposed on” is used to mean that the informational image 120 is applied to, formed on, or otherwise provided with the pull-on article 20. For example, the informational image 120 may be printed directly on the article 20 or an element thereof, or printed on a separate substrate, such as an auxiliary layer of material that is affixed or otherwise joined to the article 20 (either before or after activation). Such an auxiliary layer may be formed of non-woven, film, laminate, or other material. The informational image 120 may be applied using any known method, including printing. As used herein, the term “print” includes all printing methods as known in the art, including, but not limited to, digital, ink jet, gravure, screen, and other forms of printing. Regardless of the printing method, the resulting printed image is preferably sufficiently dry and water steadfast to resist transfer in response to dry insults (e.g., abrasion due to contact with outer clothing) and to resist transfer, run, or bleeding in response to contact with liquids (such as water, urine, or drinks).

In certain situations, it may be preferable to provide an image that is legible or more legible when the side panels are in the relaxed state. An example of such a situation is when a child is asked to perform all or a portion of the pull-on article placement. To the extent any assistance is provided, the caregiver will often help only to place a child's feet through the leg openings of the article (i.e., thread the legs through the article). Accordingly, when first confronted with the task of pulling up the article, the article is at the child's ankles or knees. In the ankle, knee, or other position below the hips and buttocks, the side panels are in a relaxed or substantially relaxed state. For example, the side panels rarely exceed 40% extension, are typically at 0-20% extension, and are often at 0-10% extension when the article is positioned below the hips and buttocks. Conversely, once the article is in place about the waist, the child may no longer need to grasp the article and/or apply a pulling force, and thus may not need to view the article or any image disposed thereon, and image legibility and visibility is less important. For this type of situation, therefore, the informational image 120 is preferably readily observable when the side panels are substantially in the relaxed state.

In other situations, it may be preferable to provide an image that is legible or more legible when the side panels are in the extended states. For example, the caregiver may apply the product to the user's hips or waist and ask the child to complete the application process, such as by pulling completely up to the waist, adjusting the fit, etc. In these circumstances, the side panels will be in a highly or substantially extended state. Accordingly, the informational image is preferably legible or more legible when the side panels are in the extended state to provide information to the user at the appropriate point of the application process.

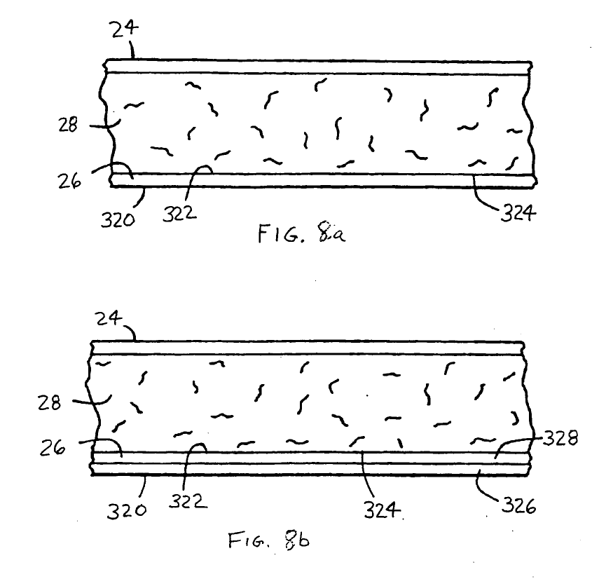

If image legibility in the relaxed state is a concern, the informational image may be disposed on the side panel with the side panel in the relaxed or substantially relaxed state. Examples of informational images that are legible or more legible in the relaxed state are illustrated in FIGS. 9-11.

FIGS. 9A and 9B illustrate one example where an image 111 is disposed on an outer layer of non-woven material 112 of a zero stretch laminate in the relaxed and extended states, respectively. The image 111 is distinct, clear, and continuous (and hence legible) when the non-woven layer 112 is in the relaxed state (FIG. 9A). With the non-woven layer 112 stretched in the extended state, discontinuities interrupt the image, making it less legible.

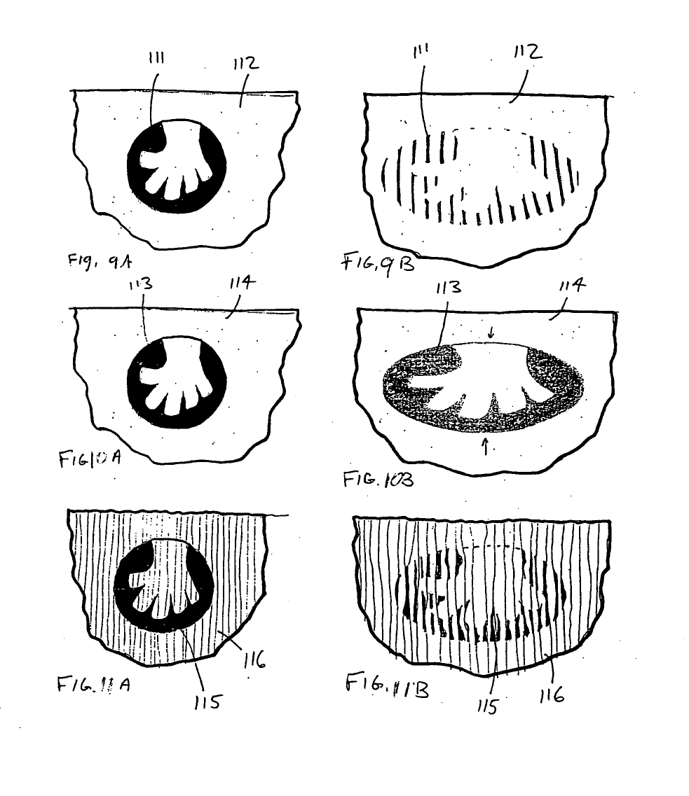

Another example is illustrated in FIGS. 10A and 10B, where an image 113 is shown on an elastomeric film layer 114 in the relaxed and extended states, respectively. When the layer 114 is in the relaxed state (FIG. 10A), the image 113 is more clear and distinct, provides greater contrast with the background color, and is less distorted, thereby providing a legible image. When the layer 114 is in the extended state shown in FIG. 10B, the image 113 has a lighter, less definite, and more distorted appearance.

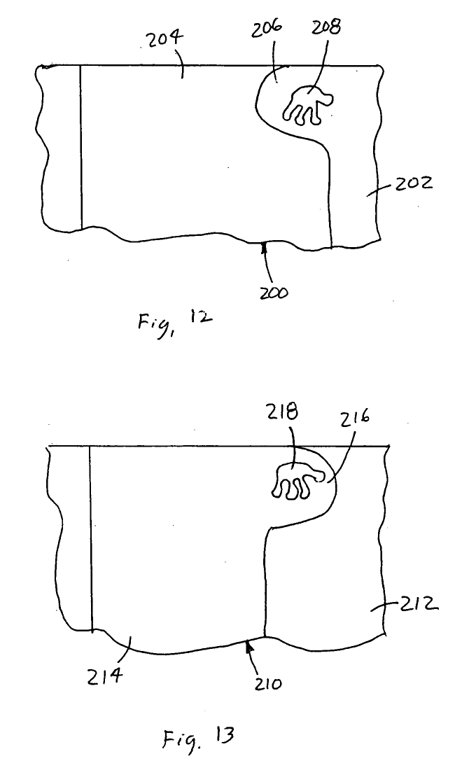

A further example is shown in FIGS. 11A and 11B, which illustrate an image 115 disposed on an outer layer of a pre-stretch laminate 116. With the laminate 116 in the relaxed state (FIG. 11A), the image 115 again is more clear, defined, and continuous, whereas the image 115 is discontinuous and more difficult to comprehend when the laminate is in the extended state illustrated in FIG. 11B.

In each of the foregoing examples, the informational image may be applied with the side panel in either the relaxed state (either before or after activation) or in the extended state. If the side panel is in the relaxed state, the image is applied as an undistorted image that substantially corresponds to the image as it is intended to be viewed by a user, such as the images shown in FIGS. 9A, 10A, and 11A. If the image is applied with the side panel in the extended state, the image is applied as a distorted image, such as those shown in FIGS. 9B, 10B, and 11B, so that the image is undistorted and enhanced when the side panel is subsequently relaxed.

It will be appreciated, however, that the informational image may be legible or more legible in the extended state without departing from the scope of this disclosure. Accordingly, an undistorted image may be applied while the side panel is in the extended state or a distorted image may be applied while the side panel is in the relaxed state.

The informational images disclosed herein are particularly suited for pull-on articles intended for use by children. Accordingly, the exemplary images illustrated herein are cognitively functional to a pre-literate child. The informational images preferably use symbols, graphics, or other markings other than words as the primary form of communication, so that a pre-literate child may comprehend and follow the instructions or other information indicated by the image.

The image may be in the form of any visual representation suitable for communicating information regarding the use of article 20 to a user. Accordingly, the image may include one or more icons, which may comprise, but are not limited to, pictorial symbols, photographs, drawings, cartoons, and logos. For example, the icons may be provided as drawings of a child or an anthropomorphic image of an animal using the pull-on article 20. Similarly, the icons may include well-known cartoon characters or brand logos, or characters specifically created to be associated with the article. The icons may further include symbols, such as arrows, to indicate motion, movement, or directionality.

The informational image may be arranged in any manner as long as it communicates the desired information to a user. The image may be a single icon or a series of icons. If a series of icons is provided, each icon may be the same or different. Different icons may be complementary to one another, in that they are related to the same concept or activity (such as open and closed hands), or incorporate a common visual element (such as a similar appearance, color, or theme). The icons may be arranged in any suitable fashion, such as, but not limited to, vertically, horizontally, diagonally, circular, arcs, and combinations thereof.

The informational image may optionally include a character graphic that can increase a user's interest in the product. In the illustrated embodiments, the informational image 120 communicates a location in which to grip the article 20 as it is pulled into place on a user. In an article having extensible side panels, forces are more effectively transferred from radial side locations to the front and back regions than from the front of the article. Accordingly, the informational image 120 is located in a side region 31 or 32 of the article 20, which include these radial side locations. It is preferable to pull the diaper at both side regions, and therefore informational images 120 may be provided in both side regions. Furthermore, it is preferable to grip the article near an upper edge, and therefore the images 120 may be positioned in an upper portion of each side panel. While the images may be different, they are preferably substantially similar. As used herein, “substantially similar images” include identical images, mirror images, images incorporating common visual elements, the same or similar image shapes having different colors, inverted foreground and background images (i.e., the same or similar image in positive and negative), the same or similar image in both solid and outline, and the like.

In an exemplary embodiment of a cognitively functional graphic, the informational image 120 is provided as a hand graphic to communicate to a user that the article is to be grasped in the location of the image. As used herein, the phrase “hand graphic” refers to an image formed to resemble a hand, a hand with a portion of an arm or body, or one or more portions thereof, such as a palm, one or more fingers, one or more fingertips, and the like. The hand may resemble a human, animal, anthropomorphic, cartoon character, mythical creature, or other style. Furthermore, the hand image is not limited to including five fingers (i.e., four fingers and a thumb). When intended for use by children, the image 120 may have any size, but is preferably sized to generally correspond to the size of a child's hand or a portion thereof. Accordingly, the image preferably has a lateral dimension of approximately 1 to approximately 10 centimeters and a longitudinal dimension of approximately 1 to approximately 5 centimeters. In an exemplary embodiment, the image may have a lateral dimension of approximately 6 centimeters and a longitudinal dimension of approximately 3 centimeters.

Graphics other than the image of a hand may be used to attract a user's attention and indicate a gripping location. When the article is intended for use by a child, it may carry images of a cookie or other foods, a spoon, a doorknob, a handle, or other object commonly grasped by a child. Alternatively, the indicia may include images of stars, balloons, or other items easily recognized by a child, or patterns and decorative designs that would attract a child's attention.

While the informational images 120 illustrated in FIG. 1a are positioned entirely within a respective side panel, the images may be located at other positions within the side panels 31, 32. For example, part of the image may be located on a side panel while another part may be located on an adjacent transverse region of the main portion. The information image, for example, could be positioned near a front waist region, or positioned near the rear waist region or at any other desired location.

In addition, the image may be located entirely within a front or rear transverse region of the main portion. The transverse regions are located adjacent to the side panels so that, when an image is provided entirely within a transverse region, it still indicates a more desirable pulling location than the center of the main portion. While a continuous, unitary side panel is illustrated in FIG. 1a, the side panel may be formed of separate panel portions. Accordingly, the image may be located entirely within a particular side panel portion, span adjacent joined side panel portions, or span a side panel portion and a transverse region of the main portion.

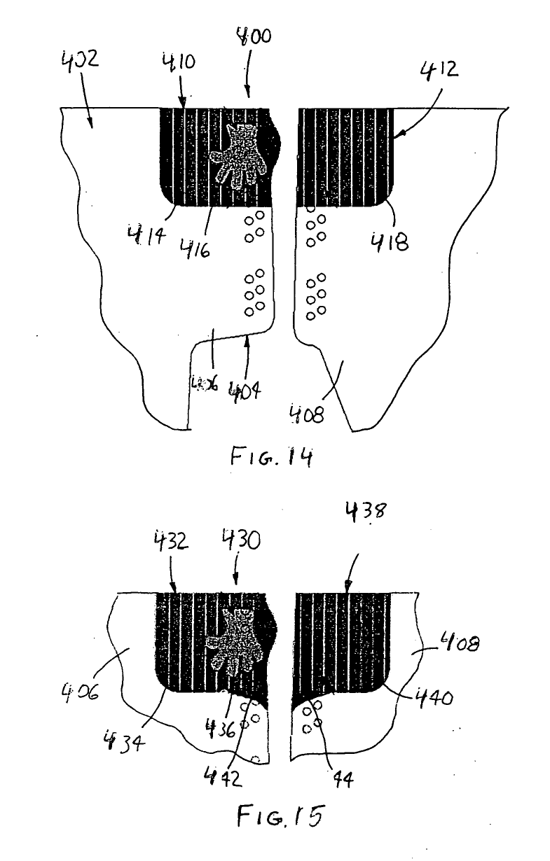

The side panel or the main portion may include a projection, such as a lateral projection, on which at least a portion of the informational image is disposed. As illustrated in FIG. 12, an article 200 includes a main portion 202 and a side panel 204. The main portion 202 includes a projection 206 extending into the side panel area. An informational image 208 is disposed on at least a portion of the projection 206. The main portion 202 may comprise a plurality of layers of material, and the projection 206 may comprise one or more of those layers. For example, the main portion 202 may comprise a film layer that forms the projection 206. In the alternative embodiment illustrated in FIG. 13, an article 210 includes a main portion 212 and a side panel 214. The side panel 214 includes a projection 216 extending into the transverse region of the main portion 212, and an informational image 218 is disposed on at least a portion of the projection 216. The side panel 214 may comprise a plurality of material layers, and the projection 216 may comprise one or more of those layers.

Whether the side panel is unitary or formed of separate panel portions, seams may be formed over which the image may be applied. Seams are formed when adjacent edges of material are bonded together over at least a portion of the adjacent edges of material to permanently join the materials. The attachment of the panel portions is permanent in the sense that the panel portions are intended to maintain a joined relationship prior to and during use. The panel portions may, however, be frangible or frangibly connected to facilitate removal and/or disposal of the article. In an article having unitary side panels, seams may be formed where the side panel joins the front and rear waist regions. Similarly, a seam may be formed between separate side panel portions that are joined together. The informational image may be formed over any seam formed in the side regions. For example, the panel portions may be joined to form a seam, and the image may span the seam.

FIG. 14 illustrates an informational image 400 spanning a seam of an article 402. The article 402 includes a side panel 404 including a front panel portion 406 and a rear panel portion 408. The front and rear panel portions 406, 408 may be joined, such as by bonding, to form a seam, however FIG. 14 shows the panel portions prior to being joined. The informational image 400 is disposed on the side panel 404 and includes a front image portion 410 and a rear image portion 412. In the illustrated embodiment, the front image portion 410 includes a background graphic 414 and a hand graphic 416, while the rear image portion includes a background graphic 418. When joined together, a peripheral region of the front panel portion 406 overlies a peripheral region of the rear panel portion. In this embodiment, the hand graphic 416 is disposed entirely on the front panel portion 406, so that the subsequently formed seam does not disrupt the image. The hand graphic 416 may, alternatively, be positioned entirely on the rear panel portion 408, or may have portions disposed on both the front and rear panel portions 406, 408.

FIG. 15 illustrates an informational image 430 similar to that shown in FIG. 14, but with a slight variation. Specifically, the informational image 430 includes a front image portion 432 including a background graphic 434 and a hand graphic 436 and a rear image portion 438 including a background graphic 440. The background graphics 434, 440 include curved or contoured lower borders 442, 444 near the lateral edges of the front and rear panel portions 406, 408. Since the image portions 432, 434 may be applied with the article in a flat configuration prior to joining the side panel portions 406, 408, there is a possibility that the image portions 432, 434 may not precisely align with one another. The curved lower borders 442, 444 make any such misalignment less readily visible, thereby allowing for greater tolerances for longitudinal tracking between side panel portions.

Rather than a single icon, the image may include a plurality or series of icons in one or more of the side regions. For example, each side panel may define an upper edge and a lower edge with a longitudinal length extending from the upper edge to the lower edge. The informational image may extend substantially across the longitudinal length of the side panel. The informational image may include a repeating pattern of hand icons. Whether a single or multiple icons are provided, the image is preferably registered so that it is located in substantially the same location on each article, however non-registered images are suitable in certain alternate embodiments.

The informational image 120 is preferably viewable from an exterior of the article, and therefore the informational image is preferably disposed on an outer or garment-facing layer of the article or an element thereof. In most cases, the image is disposed on an exterior layer of the article, such as the outer surface of the backsheet, or an auxiliary layer that is coupled to an exterior layer of the article. Alternatively, the informational image may be disposed on an interior layer and is viewable through one or more transparent or translucent outer layers. In certain alternate embodiments, the image may be viewable from the interior of the article such as by printing on an interior surface or on a layer that is visible from an interior of the article. Accordingly, the informational image may be disposed such that is viewable from an exterior only, from both an exterior and an interior, or from an interior only of the article.

In accordance with additional aspects of this disclosure, a pull-on wearable article may be provided having an informational image incorporating a texture feature. The texture feature is positioned sufficiently proximate the informational image so that the texture feature is associated with the informational image, thereby to form a composite image. As used herein, the term “proximate” includes coincident with, partially coincident with, adjacent to, or in the vicinity of one another.

The texture feature imparts a unique visual appearance to the image, such as by forming layers, regions of relative smoothness or roughness, varying reflectivity, color enhancements, or other visual effect. The texture feature may increase the legibility of the image in poor lighting conditions or at viewing angles deviating significantly from the perpendicular. Additionally or alternatively, the texture feature may enhance at least a portion of the informational image or may form a separate portion of the informational image. For example, the informational image may form a part of a hand image, such as a palm, while the texture feature forms another part of the hand image, such as fingers or finger pads, so that the combination of the informational image and the texture feature form a composite image. Alternatively, the informational image may form a complete hand image while the texture feature is applied to part or the entire hand image to form the composite image. The combination of the informational image and texture feature may be provided on any known type of pull-on wearable article having extensible side panels, without regard to the type, or method of construction of the side panels, and regardless of the state (i.e., extended or relaxed) in which the informational image is more legible, if any.

In one exemplary embodiment, the texture feature may include localized projections formed in an outer surface of the article. FIG. 16 illustrates a cross-sectional view of a portion of an article carrying an informational image. The article at this portion includes an outer layer 150 of non-woven material, an inner layer 152 of non-woven material, and an elastomeric element 154 disposed between the outer and inner layers 150, 152. The outer layer 150 is formed with localized projections 156 defining recesses 158 therebetween. The projections 156 may be formed by embossing or other processes that raise portions of at least the outer layer.

In an alternative embodiment, the texture feature may include localized recesses formed in the outer surface of the article. FIG. 17 illustrates a cross-sectional view of a portion of an article having an outer layer 160 of non-woven material, an inner layer 162 of non-woven material, and an elastomeric element 164 disposed between the outer and inner layers 160, 162. The outer layer 160 is formed with localized recesses 166 defining projections 168 therebetween. The recesses may be formed in any known manner, including application of a bond pattern between the outer layer 160 and either the inner layer 162 or elastomeric element 164. The bond pattern secures discrete locations of the outer layer 160 to one of the other interior layers to form an associated recess 166.

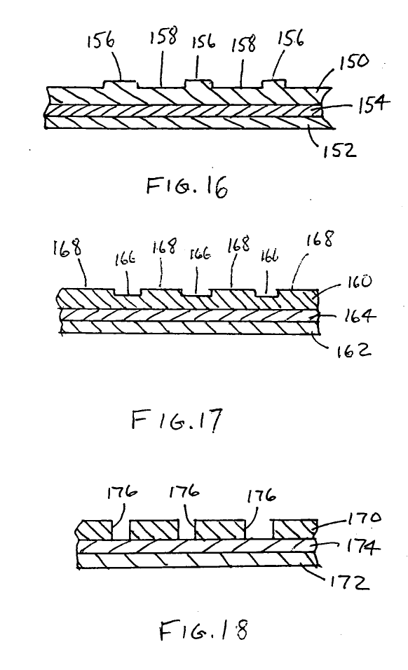

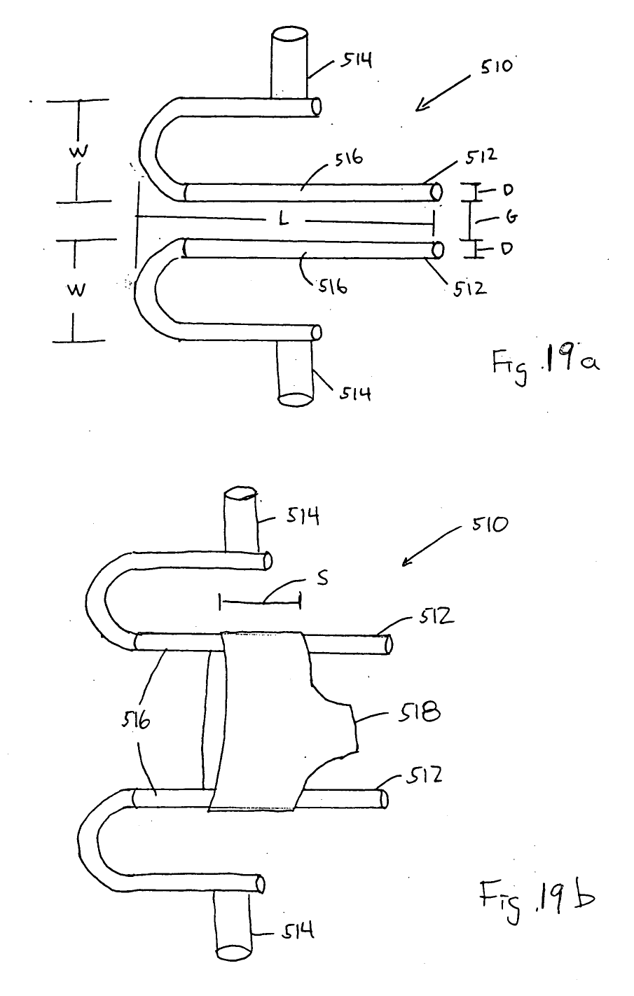

In a further exemplary embodiment, the texture feature may be formed by adjacent layers of material. As illustrated in FIG. 18, a portion of a pull-on article includes an outer layer 170 of non-woven material, an inner layer 172 of non-woven material, and an elastomeric element 174 disposed between the outer and inner layers 170, 172. The outer layer 170 is formed with slits, slots, holes, or other regular or irregular apertures or openings 176 to expose portions of the elastomeric element 174. Accordingly, it will be appreciated that the exterior surface of the article, at least in this location, is formed by both the outer layer 170 and the exposed portions of the elastomeric layer 174. The outer layer 170 has a thickness sufficient to create a varying exterior surface appearance.

While the exemplary embodiments of texture features disclosed herein identified specific layers and types of material, it will be appreciated that other combinations and types of material layers may be used. Furthermore, while specific processes for forming the texture feature are suggested, any type of known process for forming the features may be used. While the texture feature is preferably formed by a mechanical treatment (such as embossing, ring-rolling, bonding, scoring, puncturing, or slitting), it may also be formed by non-mechanical treatments such as laser, hot air, chemical, or other processes.

Each of the mechanical treatments may result in particular types of texture effects. Embossing may be performed either hot or cold, with either a smooth or a patterned roll, and may result in projections, recesses, areas of relative smoothness, areas of compression (and associated compression resistance), or combinations thereof. Ringrolling may result in openings, projections, recesses, or combinations thereof. Methods for forming structural elastic-like film (SELF) may be employed, such as those disclosed in U.S. Pat. No. 5,554,143 issued to Roe et al. on Sep. 10, 1996 entitled “Absorbent Article with Multiple Zone Structural Elastic-Like Film Web Extensible Waist Feature” and U.S. Pat. No. 5,518,801 issued to Chappell et al. on May 21, 1996 entitled “Web Materials Exhibiting Elastic-Like Behavior,” and may result in projections, recesses, or combinations thereof. Scoring may cause surface morphology such as areas of relative roughness or fuzziness. Puncturing may cause openings at least partially surrounded by three-dimensional projections.

The texture effect may further be influenced or at least partially formed by the elastomeric layer. For example, an elastomeric element having a discontinuous surface, such as the vacuum formed elastomer disclosed in U.S. Published Application No. US2003/0120240 to Buell et al. published on Jun. 26, 2003 and entitled “Disposable Pant-Type Diaper Having Improved Protection Against Red Marking,” may be provided against which the outer cover at least partially conforms, to form a layered outer surface.

Still further, the texture feature may be formed by locally contracting discrete regions of the article. This may be accomplished by a mechanical process or by another process such as heat shrinking.

The use of texturing as described above, or other suitable techniques (such as increased stiffness or bulk in localized areas) may be employed in combination with informational graphics to create “handles” on the absorbent article. Such handles could simply represent areas which are easier to grab (such as by being thickened or stiffened), or they could actually offer some assistance in transmitting pulling forces (such as with loops, hooks, protruding structures, or the like. The handles need not be, but may be highlighted by the presence of informational graphics such as those described herein.

Often, disposable absorbent articles such as absorbent article 20 are packaged in a compressed state in a package. Typically, if such articles are provided with side panels or side ears, such panels or ears will be folded or tucked under the main body portion prior to compression and packing. If the side panels of such an absorbent article 20 are provided with information graphics, this means that such informational graphics 120 may not be visible upon removal from the package. Additionally, it may be desirable to provide the package itself with at least a partially clear or substantially transparent portion allowing some of the absorbent article 20 (and in particular the informational graphics 120) to be perceived through such portion. This substantially transparent portion may the form of a transparent window, for example, though which graphics, such as informational graphics 120 may be perceived even which the articles are still in the package. In order to facilitate such viewing, it may be desired to package absorbent articles 20 in a package with the side panels 31, 30 in an un-tucked or outwardly disposed configuration. Even if articles 20 are not packaged in a package with a clear window, such an presentation in the package with the side panels viewable upon removal (without having to un-tuck or unfold them) may be desired so that the user immediately can see the informational graphic 120 without additional manipulation of the article 20 being required.

In certain suitable embodiments, ease of application and removal of absorbent articles of the invention may be accomplished through manipulation of features such as the elastic belt 70, 370. The elastic belt 70, 370 can be manipulated to provide such possible desired characteristics as percent recovery, open time, elongation force, and fit force. These desired characteristics may be achieved by varying the physical and compositional structure of the elastic belt 70, 370. In certain suitable embodiments, the elastic belt 70, 370 may comprise an elastic member exhibiting slow recovery characteristics. In particular embodiments, the side panels, waist feature, and/or waistband may comprise elastic members exhibiting slow recovery characteristics. An elastomer exhibits slow recovery characteristics if the material exhibits at least about 20% post elongation strain after 15 seconds of recovery at 22° C. as measured by the Post Elongation Recovery Test provided below.

A number of elastomeric polymers can be used to prepare an elastic material exhibiting slow recover characteristics. Elastomeric polymers include, but are not limited to, homopolymers (e.g., crosslinked poly(isoprene)), block copolymers, random copolymers, alternating copolymers, and graft copolymers. Suitable elastomeric polymers comprise styrenic block copolymers, natural and synthetic rubbers, polyisoprene, neoprene, polyurethanes, silicone rubbers, hydrocarbon elastomers, ionomers, and the like.

In one embodiment, the elastomeric polymer may be a block copolymer. A number of block copolymers may be used to prepare the elastic material exhibiting slow recovery characteristics including multi-block, tapered block and star block copolymers. Generally, the block copolymers suitable for use in the slow recovery elastomer may exhibit both elastomeric and thermoplastic characteristics. In such block copolymers a hard block (or segment) may have a glass transition temperature (Tg) greater than about 25° C. or is crystalline or semicrystalline with a melting temperature (Tm) above about 25° C. Preferably, the hard block has a Tg greater than about 35° C. or is crystalline or semicrystalline with a Tm above about 35° C. The hard block portion is typically derived from vinyl monomers including vinyl arenes such as styrene and alpha-methyl-styrene or combinations thereof.

Glass transition temperatures referred to herein with reference to elastomeric polymers and the slow recovery elastomer of the present invention are determined by tensile dynamic mechanical analysis performed in the linear elastic region of the material at a frequency of 1 Hz using a temperature ramp method. Suitably, film samples with a uniform thickness of about 0.3 mm or less may be used with a temperature ramp rate of about 1° C./min or slower. The tan δ peak temperature is taken as the Tg of the particular material or phase. Crystalline melting temperatures referred to herein are determined by Differential Scanning Calorimetry using a temperature ramp rate of 10° C./min. The melting endothermic peak temperature is taken as the Tm of the particular crystalline region.

The soft block portion may be a polymer derived from conjugated aliphatic diene monomers. Typically, the soft block monomers contain fewer than about 6 carbon atoms. Suitable diene monomers include butadiene, isoprene, and the like. Suitable soft block polymers include poly(butadiene) and poly(isoprene). Suitable block copolymers for use in this invention may comprise at least one hard block (A) and at least one soft block (B). The block copolymers may have multiple blocks. In a preferred embodiment, the block copolymer may be an A-B-A triblock copolymer, an A-B-A-B tetrablock copolymer, or an A-B-A-B-A pentablock copolymer. Also, useful herein are triblock copolymers having endblocks A and A′, wherein A and A′ may be derived from different vinyl compounds. Also, useful in the present invention are block copolymers having more than one hard block and/or more than one soft block, wherein each hard block may be derived from the same or different monomers and each soft block may be derived from the same or different monomers.

It should be noted that where the copolymer contains residual olefinic double bonds, the copolymer may be partially or fully hydrogenated if desired. Saturation may often yield beneficial effects in the elastomeric properties of the copolymer.

The elastomeric polymer may be used in the slow recovery elastomer in an effective amount so as to achieve the desired normalized unload forces and post elongation strains. The slow recovery elastomer generally may comprise from about 20% to about 70%, preferably about 30% to about 65%, and most preferably about 45% to about 60% of the elastomeric polymer.

Elastomeric polymers may include styrene-olefin-styrene triblock copolymers such as styrene-butadiene-styrene (S-B-S), styrene-ethylene/butylene-styrene (S-EB-S), styrene-ethylene/propylene-styrene (S-EP-S), styrene-isoprene-styrene (S-I-S), hydrogenated polystyrene-isoprene/butadiene-styrene (S-IB-S), and mixtures thereof. The block copolymers may be employed alone or in a blend of block copolymers. Suitable S-B-S and S-I-S copolymers are commercially available under the trade designation Vector® from Dexco Polymers L.P., Houston, Tex., and under the trade designation Kraton from Kraton Polymers, Houston, Tex.

Various modifying resins may be used in this slow recovery elastomer. Suitable modifying resins should preferably associate or phase mix with the soft blocks of the elastomeric polymer. While not intending to be bound by this theory, it is believed that the modifying resins raise the Tg of the soft phase to the point where molecular relaxation at the in-use temperature is slowed. The slow recovery elastomer may comprise the modifying resin in amounts from about 0% to about 60% by weight. Preferably, the composition comprises from about 20% to about 55% and even more preferably from about 40% to about 50% of the modifying resin. Suitable modifying resins useful herein may include, but are not limited to, unhydrogenated C5 hydrocarbon resins or C9 hydrocarbon resins, partially and fully hydrogenated C5 hydrocarbon resins or C9 hydrocarbon resins; cycloaliphatic resins; terpene resins; polystyrene and styrene oligomers; poly(t-butylstyrene) or oligomers thereof; rosin and rosin derivatives; coumarone indenes; polycyclopentadiene and oligomers thereof; polymethylstyrene or oligomers thereof; phenolic resins; indene polymers, oligomers and copolymers; acrylate and methacrylate oligomers, polymers, or copolymers; derivatives thereof; and combinations thereof. Preferably, the resin is selected from the group consisting of the oligomers, polymers and/or copolymers derived from: t-butylstyrene, cyclopentadiene, iso-bornyl methacrylate, methyl methacrylate, isobutyl methacrylate, indene, coumarone, vinylcyclohexane, methylstyrene, and 3,3,5-trimethylcyclohexyl methacrylate. Preferred modifying resins also include alicyclic terpenes, hydrocarbon resins, cycloaliphatic resins, poly-beta-pinene, terpene phenolic resins, and combinations thereof. “C5 hydrocarbon resins” and “C9 hydrocarbon resins” are disclosed in U.S. Pat. No. 6,310,154.

In other suitable embodiments, the elastic belt may comprise an elastic member that is a heat shrinkable material, a water shrinkable materials, a memory visco-elastic foam, a plasto-elastic material, or combinations thereof.

Any of the above described product features can be combined in any desired combination. For example, product features including (but not limited too), a wetness sensation member (either permanent or removable), refastenable sides, appearing graphics, informational graphics (such as hand print graphics), and slow recovery elasticized portions of the article may all be combined in a single disposable pant like garment product offering. In other variations, only some of these features may be included, and they may offered in any desired combination or sub-combination. Additionally, variations of products may be combined into a single package to provide a potty training kit or system, and particular suggestions of model systems may be provided. As an example, a series of pant like garments may be include in a single potty training system kit. Some of the products might have a wetness sensation liner, and others may not. In such an example, other product features (such as refastenable sides and appearing graphics) may be included in all products in the kit, in none of them, or in some products in any desired combination.

Test Methods

Percent Release and Percent Maximum Force

This method is used to measure the force exerted by a pull-on garment at a point of recovery after elongation. The force data may be used to calculate a Percent Maximum Force, which is a measure of the percent of the maximum force that is exhibited at some given point in time. The force data may be used to calculate a Percent Release, which is a measure of the change in force from some point in time compared to some later point in time. Time values are measured starting from the point in time when the gauge length, but not necessarily the sample, reaches 30% strain upon recovery from 80% strain. The zero time point corresponds to step 6 in the tensile tester program provided below.

The percent release test is performed on a tensile tester at a constant rate of extension with a computer interface. An exemplary tester is an MTS Synergy tensile tester interfaced with Testworks 4 software. The test is conducted at ambient room condition with a temperature of 23° C.±1° C. and a relative humidity of 50%±2%. For this test, the tensile tester is fitted with a 100N load cell and custom hook fixtures 510 as shown in FIG. 19a.

The hook fixture 510 comprises a pair J-shaped hooks 512 each with an attachment member 514. Each J-shaped hook 512 has a substantially circular cross-sectional shape with a diameter, D, of about 1 cm. The hook may have a length, L, of about 20 cm. The hook may have a width, W, of about 6 cm. The hooks 512 exhibit a smooth curvature to form the two arms that are substantially parallel to one another. The hooks 512 are formed from a material that will not interfere with the measurement of the absorbent article such as Teflon-coated steel. Each hook 512 has an attachment member 514 that may be used to attach the hook to the tensile tester. Appropriate dimensions of the attachment member 514 may be varied to meet the needs of the tensile tester used. An engaging arm 516, the portion of the hook 512 that engages the sample, may be pivotally attached to the rest of the hook 512 such that the engaging arm 516 may rotate about its axis, which is the center of its cross-sectional face. The distance between the J-shaped hooks 512 is the gauge length, G.

The sample is measured to the nearest millimeter along the sample's waist edge to determine the circumference of the waist opening of the article. The initial gauge length G of the tensile tester is set to half of this circumference.

The sample 518 is loaded onto the hooks 512 as depicted in FIG. 19b. The sample 518 is positioned so that the sample's waist edge is positioned perpendicular to the engaging arms 516 of the J-shaped hooks 512. The J-shaped hooks 512 may be inserted into the waist opening. The sides of the sample (e.g., side panel, if present) should be adjacent to the J-shaped hooks 512. The sample is slid onto the J-shaped hooks until the hooks exit the opposite side of the sample. For a pull-on diaper, one hook should enter the waist opening and exit a first leg opening and the other hooks should enter the waist opening and exit the second leg opening. The sample width, S, is the width of the portion of the sample that is in contact with the J-shaped hooks measured to the nearest millimeter.

The tensile tester may be programmed as follows:
1. The time channel is set to zero. The load channel is set to zero.
2. The gauge length is extended to 80% strain (i.e., initial gauge length is extended to a length equal to 1.8 times the initial gauge length) at a crosshead speed of 508 mm/min.
3. The sample is held at 80% strain for 10 seconds.
4. (Optional) If the sample needs to be activated, a triggering event that activates the sample is applied at the end of step 3. For example, if the sample contains a heat shrink film, sufficient heat may be applied upon the end of the 10 second hold of step 3.
5. The gauge length is reduced to 30% strain (i.e., reduced to the length equal to 1.3 times the initial gauge length) at a crosshead speed of 508 mm/min.
6. Upon return to 30% strain, the time channel is again returned to zero and force values may be recorded (see Table 1 below). The gauge length is maintained at 30% strain for three minutes.
7. Force values are recorded and plotted versus time.

Percent Release is a measure of the percent change in force over a specified unit of time. Percent release may be calculated according to the formula below where n is a time greater than 1 second:

$$Percent {Release}_{time = n} = \frac{( {Force}_{time = n} - {Force}_{1 second} )}{{Force}_{time = n}} \times 100$$

In the Percent Release calculation, times are measured from the point where the gauge length reaches 30% strain as in Step 6 above. For example, Force_{1 second} is the force recorded 1 second after the time channel is zeroed upon return of the gauge length to 30% strain.

Percent Maximum Force is a measure of the percent of the maximum force that is exhibited at some given point in time. For purposes of this measure, the term “maximum force” is the force measured at 180 seconds after the time channel has been reset to zero in Step 6 according to the Test Method presented above. Percent Maximum Force may be calculated according to the formula below where n is time:

$$Percent Maximum {Force}_{time = n} = \frac{{Force}_{time = n}}{{Force}_{time = 180 seconds}} \times 100$$

30% Recovery Time

The 30% Recovery Time is a measure of the time it takes for a pull-on diaper to return to 30% strain after release from an elongation of 80% strain. For purposes of this calculation, the sample is considered to have returned to 30% once a measurable force is exerted by the sample and recorded by the tensile tester (Step 7 in the method provided above). The 30% recovery time is the time at which a force is exerted by the sample onto the hooks. The 30% Recovery Time can be considered a quantitative measure of the qualitative phenomena of “snap-back.” Samples exhibiting low 30% Recovery Times may be considered fast in that the sample recovers to 30% strain instantaneously (i.e., generally considered a time less than about 1 second) after release from an elongating force at 80% strain. Conversely, samples exhibiting higher 30% Recovery Times can be considered slow in the recovers to 30% strain over time (i.e., generally considered a time greater than about 1 second). Furthermore, since time zero begins once the gauge length returns to 30% strain, a sample that exhibits a force at time zero may be considered to have recovered at least as fast as that of the gauge speed of the tensile tester. Since the gauge speed on return from 80% strain to 30% strain is 508 mm/min, samples exhibiting a force at time zero are considered to exhibit a recovery speed of 508 mm/min or faster.

Post Elongation Recovery Test Method for Elastomers

This method is used to determine the post elongation strain of an elastomer as a function of temperature and time. This method includes stretch method and a recovery method. The measurement may be done at 22° C. (72° F.) or at 32° C. (90° F.). The method employs a Dynamic Mechanical Analyzer (DMA) such as a TA Instruments DMA 2980 (hereinafter “DMA 2980”), available from TA Instruments, Inc., of New Castle, Del.; equipped with a film clamp, Thermal Advantage/Thermal Solutions software for data acquisition, and Universal Analysis 2000 software for data analysis. Many other types of DMA devices exist, and the use of dynamic mechanical analysis is well known to those skilled in the art of polymer and copolymer characterization.

Methods of operation and calibration and guidelines for using the DMA 2980 are found in TA Instruments DMA 2980 Operator's Manual issued March 2002, Thermal Advantage User's Reference Guide issued July 2000 and Universal Analysis 2000 guide issued February 2003. To those skilled in the use of the DMA 2980, the following operational run conditions should be sufficient to replicate the stretch and recovery of the samples.

The DMA 2980 is set to the Controlled Force Mode with the film clamp. The film clamp is mounted onto the DMA 2980 and calibrated according to the User's Reference Guide. The material to be tested is cut into samples of substantially uniform dimension. Appropriate sample dimensions may be selected to achieve the required strain. For the DMA 2980, suitable sample dimensions are approximately 6.4 mm wide by approximately 0.15 mm thick. The floating film clamp of the DMA 2980 is adjusted to a position which provides approximately 6 mm between the clamping surfaces, and is locked in this position. The sample is mounted in the film clamps and the lower clamp is allowed to float to allow determination of the actual gauge length which exists between the film clamps.

Stretch Method—Specific DMA 2980 parameter settings for the above sample dimensions are set as follows: Preload force applied to sample in clamp (0.01N); auto zero displacement (on) at the start of the test; furnace (close), clamp position (lock), and temperature held at Tᵢ (22° C. or 32° C.) at the end of the stretch method. Data acquisition rate is set at 0.5 Hz (1 point per 2 seconds). The stretch method is loaded onto the DMA 2980. The method segments are (1) Initial Temperature Tᵢ (22° C. or 32° C.), (2) Equilibrate at Tᵢ (3) Data Storage ON, and (4) Ramp Force 5.0 N/min to 18.0 N.

Upon initiation of the test, the temperature ramps to the specified Tᵢ (22° C. or 32° C.) [method segment 1] and the temperature is maintained at this Tᵢ [method segment 2]. After a minimum of 15 minutes at Tᵢ, the operator initiates the sample stretching and concurrent data collection [method segments 3 and 4]. The sample is stretched with an applied ramp force of 5 N per minute to approximately 30 mm in length. The sample is locked in place at the stretched length of approximately 30 mm and maintained at Tᵢ. The force required to reach the 400% strain is recorded manually from the digital readout on the instrument.

For samples of different dimensions, the applied force is adjusted to achieve an applied ramp force of 5 N/min per square millimeter of initial sample cross-sectional area, and the maximum displacement is adjusted to achieve a strain of 400%. The percent strain is calculated by subtracting the gauge length from the stretched length, then dividing the result by the gauge length and multiplying by 100. A sample stretched from an initial length of 6 mm to a length of 30 mm results in a 400% strain.

Recovery Method—The Recovery Method is loaded onto the instrument and initiated 15 seconds after reaching the desired strain (400%) in the Stretch Method. The four segments of the recovery method are (1) Data Storage ON, (2) Force 0.01N, (3) Ramp to Tᵢ, and (4) Isotherm for 3.0 minutes. The following DMA 2980 parameter setting is changed from the Stretch Method: auto zero displacement is changed to (OFF). The Recovery Method measures the length of the sample over a 3 minute time period at the specified temperature (Tᵢ=either 22° C. or 32° C.). The sample length, percent strain, and test temperature may be recorded as a function of recovery time.

### EXAMPLES

### Examples 1 and 2

These examples are of an illustrative waistband that may be used in the diaper depicted in FIGS. 3-4. The waistband may be constructed as follows:
1) An elastomeric film may be compounded comprising about 45% Vector 4211 available from Dexco Polymers L.P., Houston, Tex., 45% poly(t-butylstyrene), and 10% mineral oil. The poly(t-butylstyrene) is ideally of approximately 12 kDa weight average molecular weight. The elastomeric film was subjected to aging. The film used in Examples 1 and 2 were aged at least 24 months
2) The elastomer film may be resized into substantially rectangular bands having a length of 120 mm, a width of 5 mm, and a thickness of about 0.14 mm to about 0.17 mm. The bands may be elongated to 600 mm (i.e., 400% strain) and bonded to a first substrate. The first substrate may be from 620-660 mm in length and may be from 70-120 mm in width, preferably 70 mm. The first substrate may be a commercial nonwoven such as supplier code H0201010 available from Fibertex A/S, Aalborg, Denmark. Approximately 14 bands of 5 mm wide elastomer film may be adhered to the first substrate by use of an adhesive such as H2031 available from Bostik Findley, Middleton, Mass. An adhesive laydown of approximately 30 g/m² on the first substrate is sufficient. The bands are evenly distributed across the width of the first substrate. In their elongated state, the bands extend substantially the length of the first substrate and are substantially parallel to the longest edge of the substrate.
3) A second substrate is bonded, by use of a second application of adhesive, to the first substrate such that the bands are positioned therebetween. Ideally, the second substrate may be the same as the first substrate (i.e., same dimensions and composition). A second application of adhesive (i.e., H2031) may be applied to the first substrate/band laminate. The resulting stretch laminate is compressed using a hand roller.
4) Two stretch laminates may be formed according to steps 1-3. The two stretch laminates may be placed is a face-to-face relationship such that the two laminates fully overlap. The two laminates are bonded to one another along their shortest distal edges. The bonding area may extend anywhere from about 5 mm to about 15 mm in width as measured along the longest edge of the laminate. The two laminates may be bonded together with an adhesive such as H2031 with a laydown of 30 g/m².
5) The two bonded stretch laminates result in a waistband having a circumference of about 300-380 mm. Example 1 had a circumference of about 306 mm. Example 2 had a circumference of about 360 mm.

An absorbent assembly may be attached to the waistband to yield an absorbent article substantially similar to the one depicted in FIGS. 3-4. Absorbent assembly construction is well known in the art. Ideally, the absorbent assembly will be bonded to the waistband so as to minimize the amount of overlap between the absorbent assembly and the waistband. Examples 1 and 2 may be constructed according to the steps provided above. Example 1 differs from Example 2 in circumference. Example 1 had a circumference of about 306 mm and Example 2 had a circumference of about 360 mm.

### Examples 3 and 4

These examples are comparative examples using a Pampers® Easy Up size 2T-3T, available from The Procter & Gamble Company, Cincinnati, Ohio.

### Example 5 and 6

These examples are comparative examples using a Huggies Pull-Ups® boys size 3T-4T, available from Kimberly-Clark Corp., Neenah, Wis.

### Example 7 and 8

These examples are comparative examples using a Huggies Pull-Ups® girls size 3T-4T, available from Kimberly-Clark Corp., Neenah, Wis.

Test Results

Provided below in Tables 1 and 2 are the results of the Percent Release Test for Examples 1-8. Table 1 lists the raw force values for the Examples at discrete points in time. Table 2 shows the forces of Table 1 normalized and rounded to the nearest 1/100^{th} decimal place. Normalization may be performed by taking the raw force values for the Examples at various points in time (as provided in Table 1) and dividing by the width of the sample. The force values are considered accurate to plus or minus 0.05 N. As a result, the negative force value for Example 1 at the time of 1 second is believed to be a signal-to-noise artifact and, given the accuracy of the measurement may correspond to no force at the time of 1 second. Likewise, the force value for Example 2 at the time of 1 second may also correspond to no force. As can be seen from the data of Table 1, the present invention (Examples 1-2) exhibits a recovery where the forces gradually build to the maximum force as measured at the time of 180 seconds. The comparative examples (Examples 3-8) show that the force values are high at the start of the measurement cycle and increases slightly after a 15-30 seconds time.

TABLE 1

| | Forces (N) | | | | | | | |
|---|---|---|---|---|---|---|---|---|
| | Time | | | | | | | |
| | 1 | 15 | 30 | 45 | 60 | 90 | 120 | 180 |
| Example 1 | −0.02 | 0.15 | 0.45 | 0.69 | 0.84 | 1.10 | 1.24 | 1.35 |
| Example 2 | 0.03 | 0.21 | 0.52 | 0.72 | 0.87 | 1.13 | 1.26 | 1.36 |
| Example 3 | 2.50 | 3.26 | 3.39 | 3.45 | 3.51 | 3.53 | 3.57 | 3.56 |
| Example 4 | 2.57 | 3.25 | 3.40 | 3.48 | 3.52 | 3.54 | 3.59 | 3.61 |
| Example 5 | 4.46 | 5.05 | 5.17 | 5.22 | 5.25 | 5.28 | 5.31 | 5.30 |
| Example 6 | 4.15 | 4.71 | 4.83 | 4.88 | 4.89 | 4.94 | 4.94 | 4.95 |
| Example 7 | 4.94 | 5.59 | 5.70 | 5.76 | 5.79 | 5.80 | 5.82 | 5.81 |
| Example 8 | 4.85 | 5.42 | 5.53 | 5.57 | 5.59 | 5.62 | 5.63 | 5.63 |

TABLE 2

| | | Forces (N/cm) | | | | | | | |
|---|---|---|---|---|---|---|---|---|---|
| | | Time (s) | | | | | | | |
| | Width (mm) | 1 | 15 | 30 | 45 | 60 | 90 | 120 | 180 |
| Example 1 | 70 | 0.00 | 0.02 | 0.06 | 0.10 | 0.12 | 0.16 | 0.18 | 0.19 |
| Example 2 | 70 | 0.00 | 0.03 | 0.07 | 0.10 | 0.12 | 0.16 | 0.18 | 0.19 |
| Example 3 | 89 | 0.28 | 0.37 | 0.38 | 0.39 | 0.39 | 0.40 | 0.40 | 0.40 |
| Example 4 | 89 | 0.29 | 0.37 | 0.38 | 0.39 | 0.40 | 0.40 | 0.40 | 0.41 |
| Example 5 | 110 | 0.41 | 0.46 | 0.47 | 0.47 | 0.48 | 0.48 | 0.48 | 0.48 |
| Example 6 | 110 | 0.38 | 0.43 | 0.44 | 0.44 | 0.44 | 0.45 | 0.45 | 0.45 |
| Example 7 | 110 | 0.45 | 0.51 | 0.52 | 0.52 | 0.53 | 0.53 | 0.53 | 0.53 |
| Example 8 | 110 | 0.44 | 0.49 | 0.50 | 0.51 | 0.51 | 0.51 | 0.51 | 0.51 |

Table 3 provides the percent maximum forces for the various examples at discrete points in time. The percent maximum force for a given time is computed by taking the force measurement at some time and dividing by the force at time=180 second and multiplying by 100. As used herein, the term “maximum force” refers to the force, either raw or normalized, measured for a sample at 180 seconds. This percent maximum force value can be used to show how quickly it takes for the sample to approach its maximum force value and relatively how much force is exerted.

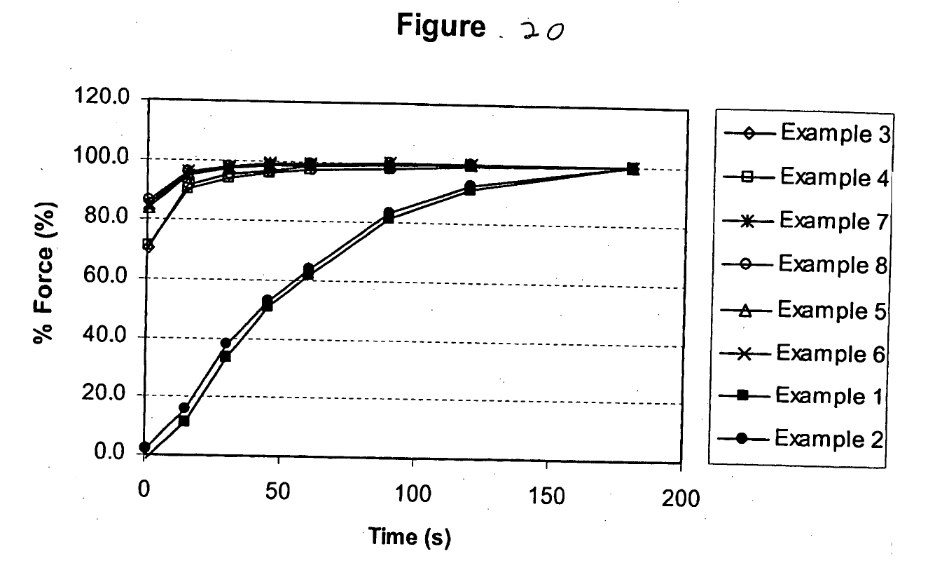

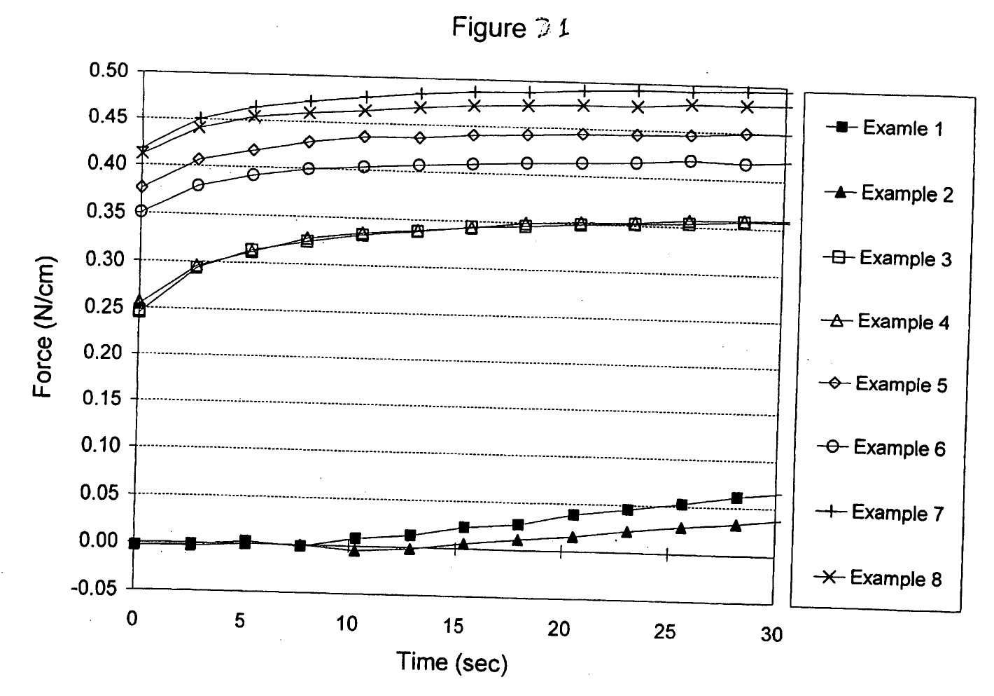

FIG. 20 is a graph that incorporates the data from Table 3 with the Percent Maximum Force being plotted versus time. As may be appreciated from Table 3 and/or FIG. 20, the present invention (Examples 1 and 2) exhibit a slow build in force to reach the maximum force. Conversely, the comparative examples (Examples 3-8) exhibit relatively instantaneous (e.g., as measured at time=1 second) exertion of a large percent (i.e., 70% of more) of the maximum force. Furthermore, by 15 seconds, the comparative examples are exhibiting at least 90% of their maximum force whereas the present invention is exhibiting only about 11-15% of its maximum force. Qualitatively, the present invention does not exhibit the “snap back” present in the comparative examples. When viewed in relation to a child self-applying a pull-on diaper, low initial force (i.e., a low percent maximum force at the lower time values) means that the child may extend the waist opening of the pull-on diaper without much effort. It is believed that a pull-on diaper exhibiting lower initial forces is easier to apply by a child with or without caregiver assistance.

TABLE 3

| | Percent Maximum Force (%) | | | | | | | |
|---|---|---|---|---|---|---|---|---|
| | Time (s) | | | | | | | |
| | 1 | 15 | 30 | 45 | 60 | 90 | 120 | 180 |
| Example 1 | −1 | 11 | 33 | 51 | 62 | 81 | 92 | 100 |
| Example 2 | 2 | 15 | 38 | 53 | 64 | 83 | 93 | 100 |
| Example 3 | 70 | 92 | 95 | 97 | 99 | 99 | 100 | 100 |
| Example 4 | 71 | 90 | 94 | 96 | 98 | 98 | 99 | 100 |
| Example 5 | 84 | 95 | 98 | 98 | 99 | 100 | 100 | 100 |
| Example 6 | 84 | 95 | 98 | 99 | 99 | 100 | 100 | 100 |
| Example 7 | 85 | 96 | 98 | 99 | 100 | 100 | 100 | 100 |
| Example 8 | 86 | 96 | 98 | 99 | 99 | 100 | 100 | 100 |

Table 4 shows the percent release values for each of the Examples. The equation for calculating Percent Release is presented above. As may be appreciated from the percent release calculation, Percent Release values approaching zero indicate that the force at time=n has not increased appreciably compared to the force at time=1 second. Likewise, a percent release value approaching 100 indicates that the force at time=n has increased appreciably compared to the force at time=1 second.

TABLE 4

| | Percent Release (%) | | | | | | |
|---|---|---|---|---|---|---|---|
| | Time (s) | | | | | | |
| | 15 | 30 | 45 | 60 | 90 | 120 | 180 |
| Example 1 | 112.6 | 104.1 | 102.7 | 102.2 | 101.7 | 101.5 | 101.4 |
| Example 2 | 83.7 | 93.5 | 95.3 | 96.1 | 97.0 | 97.3 | 97.5 |
| Example 3 | 23.3 | 26.2 | 27.5 | 28.6 | 29.1 | 30.0 | 29.6 |
| Example 4 | 21.0 | 24.5 | 26.2 | 26.9 | 27.4 | 28.5 | 28.8 |
| Example 5 | 11.8 | 13.8 | 14.6 | 15.1 | 15.7 | 16.1 | 16.0 |
| Example 6 | 11.8 | 14.0 | 14.9 | 15.0 | 16.0 | 15.8 | 16.1 |
| Example 7 | 11.6 | 13.2 | 14.2 | 14.6 | 14.8 | 15.1 | 14.8 |
| Example 8 | 10.5 | 12.1 | 12.9 | 13.2 | 13.7 | 13.8 | 13.8 |

FIG. 21 is a plot of the normalized forces versus time for Examples 1-8. As can be seen from the graph, the comparative examples (Examples 3-8) illustrate that a force is being applied to the hooks at time=0 seconds. As a result, Examples 3-8 each exhibit a 30% Recovery Time of 0 seconds. Conversely, as shown in FIG. 21, the present invention (Examples 1-2) exhibits a 30% Recovery Time of between approximately 7.5 seconds and 12.5 seconds. It is believed that the 30% Recovery Times exhibited by the present invention results in the waist opening maintaining an enlarged state during application of the pull-on diaper. Consequently, the 30% Recovery Time exhibited by the present invention may allow for easier application of a pull-on diaper.

All documents cited in the Detailed Description of the Invention and the Background are, in relevant part, incorporated herein by reference; the citation of any document is not to be construed as an admission that it is prior art with respect to the present invention.

While particular embodiments and/or individual features of the present disclosure have been illustrated and described, it would be obvious to those skilled in the art that various other changes and modifications can be made without departing from the spirit and scope of the disclosure. Further, it should be apparent that all combinations of such embodiments and features are possible and can result in preferred executions of the disclosure. Therefore, the appended claims are intended to cover all such changes and modifications that are within the scope of this disclosure.

What is claimed is:

1. An absorbent article comprising:
a front waist region having a pair of longitudinal sides, a rear waist region having a pair of longitudinal sides, and a crotch region connecting said front waist region and said rear waist region, a pair of front side panels extending outwardly from each of said longitudinal sides of said front waist region, a pair of rear side panels extending outwardly from each of said longitudinal sides of said rear waist region, a pair of side seams joining each of said front side panels with a corresponding rear side panel so as to form a pant-like garment having a waist opening and a pair of leg openings, said absorbent article comprising at least one application or removal aid selected from the group consisting of an informational image feature, a slow recovery elasticized region feature, and refastenable side seams, said absorbent article further comprising at least one sensory feedback feature said sensory feedback feature being selected from the group consisting of appearing graphics, disappearing graphics, and a wetness sensation member.

2. The absorbent article of claim 1 wherein said at least one application or removal aid comprises an informational image.

3. The absorbent article of claim 2 wherein said informational image communicates a pulling location in which to grip the article.

4. The absorbent article of claim 3 wherein said informational image is located on one of said front side panels and said rear side panels.

5. The absorbent article of claim 2 wherein said informational image comprises a hand graphic.

6. The absorbent article of claim 5 wherein said hand graphic has size dimensions substantially corresponding to at least a portion of a child's hand.

7. The absorbent article of claim 6 wherein said hand graphic has a lateral size dimension of about 1 to about 10 centimeters and a longitudinal size dimension of about 1 to about 5 centimeters.

8. The absorbent article of claim 1 wherein said at least one application or removal aid comprises a slow recovery elasticized region.

9. The absorbent article of claim 8 wherein said slow recovery elasticized region comprises an elastic belt region disposed generally about said waist opening.

10. The absorbent article of claim 9 wherein said elastic belt region exhibits less than about 50% of the maximum force after 15 seconds as measured by the Percent Release Test.

11. The absorbent article of claim 9 wherein said elastic belt region exhibits less than about 90% of the maximum force after 45 seconds as measured by the Percent Release Test.

12. The absorbent article of claim 9 wherein said elastic belt region exhibits a 30% Recovery Time of at least 1 second.

13. The absorbent article of claim 9 wherein said elastic belt region exhibits a recovery speed of less than about 508 mm/min.

14. The absorbent article of claim 1 wherein said at least one application or removal aid comprises a refastenable side seam.

15. The absorbent article of claim 14 wherein said refastenable side seam comprises mechanical fasteners.

16. The absorbent article of claim 15 wherein said mechanical fasteners comprise hook and loop type fasteners.

17. The absorbent article of claim 15 wherein said mechanical fasteners comprise hook to hook type fasteners.

18. The absorbent article of claim 14 wherein said refastenable side seam comprises cohesive fasteners.

19. The absorbent article of claim 14 wherein said refastenable side seam comprises adhesive fasteners.

20. The absorbent article of claim 1 wherein said sensory feedback feature comprises appearing graphics.

21. The absorbent article of claim 20 wherein said appearing graphics appear over time.

22. The absorbent article of claim 21 wherein said appearing graphics are less visible upon exposure to liquid.

23. The absorbent article of claim 1 wherein said sensory feedback feature comprises disappearing graphics.

24. The absorbent article of claim 23 wherein said disappearing graphics become less visible upon exposure to liquid.

25. The absorbent article of claim 1 wherein said sensory feedback feature comprises a wetness sensation member.

26. The absorbent article of claim 25 wherein said wetness sensation member is optionally removable from said article by a user.

27. An absorbent article comprising:
a front waist region having a pair of longitudinal sides, a rear waist region having a pair of longitudinal sides, and a crotch region connecting said front waist region and said rear waist region, a pair of front side panels extending outwardly from each of said longitudinal sides of said front waist region, a pair of rear side panels extending outwardly from each of said longitudinal sides of said rear waist region, a pair of side seams joining each of said front side panels with a corresponding rear side panel so as to form a pant-like garment having a waist opening and a pair of leg openings, said absorbent article comprising at least one application or removal aid said absorbent article further comprising at least one sensory feedback feature for toilet training.
